(19) United States
(12) Patent Application Publication
(10) Pub. No.: US 2026/0130923 A1
(43) Pub. Date: May 14, 2026

(54) SOLID DOSAGE FORM FOR THE TREATMENT OF PRIMARY BILIARY CHOLANGITIS

(71) Applicant: MILI HEALTHCARE TRADE DMCC, LIMITED LIABILITY COMPANY, Dubai (AE)

(72) Inventor: Nitin JAIN, Delhi (IN)

(21) Appl. No.: 19/441,474

(22) Filed: Jan. 6, 2026

Related U.S. Application Data
(62) Division of application No. 17/911,373.

(30) Foreign Application Priority Data
Mar. 6, 2020 (UA) U202001611
Mar. 6, 2020 (UA) U202001613
Mar. 5, 2021 (UA) A202101117

(51) Int. Cl. A61K 31/575; A61K 9/24; A61P 1/14; G01N 33/564

(52) U.S. Cl. CPC A61K 31/575; A61K 9/209; A61P 1/14; G01N 33/564; G01N 2800/085

(57) ABSTRACT

The invention relates to the field of medicine, in particular to solid dosage forms for the treatment of primary biliary cholangitis (PBC). The invention provides a modified release solid dosage form comprising two active pharmaceutical ingredients, obeticholic acid and ursodeoxycholic acid, arranged to release sequentially over time. The solid dosage form is designed to achieve a controlled, separate release of each API to provide a desired therapeutic profile.

### CROSS REFERENCE TO RELATED APPLICATIONS

This application is a Divisional of co-pending application Ser. No. 17/911,373 filed on Sep. 13, 2022, which is a U.S. National Stage of International Application No. PCT/IB2021/051918 filed on Mar. 8, 2021, for which priority is claimed under 35 U.S.C. § 120; and this application claims priority of Application No. u202001611 filed in Ukraine on Mar. 6, 2020, Application No. u202001613 filed in Ukraine on Mar. 6, 2020, and Application No. a202101117 filed in Ukraine on Mar. 5, 2021 under 35 U.S.C. § 119; the entire contents of all of which are hereby incorporated by reference.

### BACKGROUND OF THE INVENTION

### Field of the Invention

The present disclosure relates to the field of medicine, in particular, to the drugs in a solid dosage form for the treatment of primary biliary cholangitis.

### Brief Description of the Related Art

Primary biliary cholangitis (PBC), formerly known as primary biliary cirrhosis, is the most common autoimmune liver disease. This disease results in a gradual destruction of the small bile ducts of the liver, which leads to the accumulation of the bile in the liver tissues (a condition called cholestasis). Over time, the accumulated bile starts to destroy the liver, leading to fibrosis and cirrhosis. The risk group is middle-aged women, wherein the ratio of suffering women to men ranges from 1.6 to 10-14, although higher mortality rates have been recorded for men.

At first, PBC was considered a very rare disease, due to the small sample size and lack of the large population-based studies. Moreover, PBC is unevenly distributed throughout different regions of the world. The highest prevalence rates were recorded in China, the USA, Greece, and England (49.2; 40.2; 36.5 and 24.0 cases per 100,000 people, respectively), while Canada and Australia had the lowest number of the cases (2.2 and 1.9 cases, respectively). However, the overall incidence of PBC in the world has continued to rise since the 1980s.

Today, there are no studies of the epidemiology of PBC. Studies conducted in Europe and Asia generally report a steady increase in the prevalence of the disease over the past decade. In the USA, over a 12-year period, the prevalence of PBC increased by more than 72% in women (from 33.5 to 57.8 per 100,000 people), and by more than 114% in men (from 7.7 to 15.4 per 100,000 people). The Hong Kong Epidemiology Study found that the age/sex-adjusted medium annual incidence rate increased from 6.1 to 8.1 per million person-years, and the age/sex-adjusted prevalence rate increased from 31.1 to 82.3 per million person-years between 2000 and 2015. In Sweden, a large-scale population-based analysis of inpatient and outpatient registries showed that the prevalence of PBC steadily increased from 5.0 to 34.6 per 100,000 people from 1987 to 2014, respectively.

The causes and factors leading to the development of PBC are unknown. Currently, several theories are being considered, but no clear solution has been found to this issue.

One of the determinant factors for the development of PBC is the loss of immune tolerance to the autoantigen of the PDC-E2 pyruvate dehydrogenase complex. The PDC-E2 complex plays a fundamental role in activating the cellular response of Th1 T-helpers. Th1-cells start to produce INF-γ interferon gamma and TNF-α tumor necrosis factor, that have a cytotoxic effect.

The abnormal destruction of the liver cells (hepatocytes) and bile duct epithelial cells (cholangiocytes) in PBC may be explained by the hypothesis of bicarbonate (HCO₃⁻) umbrella loss, which is supported by experimental, clinical, and genetic studies. The hypothesis is based on the statement that, by secreting a bicarbonate anion (HCO₃⁻) into the lumen of the bile duct, cholangiocytes and hepatocytes create a protective apical alkaline barrier that stabilizes glycocalyx. This alkaline barrier also preserves bile acids in the form of polar, water-soluble conjugates (often also referred to as bile salts) that are unable to cross cell membranes. In PBC, the functioning of the transporters and channels located on the apical and basolateral membranes of cholangiocytes involved in the formation and export of HCO₃⁻ ions is disrupted. This results in weakening of the alkaline barrier, which leads to partial protonation of the bile acid conjugates resulting in that they become apolar and acquire the ability to cross the cholangiocyte membrane, regardless of the activity of the transporters, and induce apoptosis in cholangiocytes and hepatocytes.

Recent studies have shown that the POGLUT1 gene plays an important role in pathogenesis of this disease. Moreover, the tendency to develop PBC is associated with polymorphism in the human leukocyte antigen (HLA) region, especially in the DRB1*08 DRB1*11, DRB1*14 DPB1*03:01 and DQB1 alleles.

Additionally, over the past decades, a correlation between the etiopathogenesis of the disease, environmental factors, and impaired immune tolerance in PBC has been observed. In this regard, the role of pathogens of various infections in the etiology of PBC has been hypothesized. For example, patients with PBC have a higher prevalence of urinary tract infections, mainly associated with E. coli. It has also been shown that the gram-negative microorganism Novosphingobium aromaticivorans can be an etiological factor in the development of PBC. The theoretical mechanism is the cross-reactivity of antibodies against the surface proteins of the microorganism with mitochondria of hepatocytes.

In addition, the gut microflora can also influence PBC, since the presence of metabolic by-products of microorganisms, such as Toll-like receptor (TLR) ligands and CpG motifs can contribute to the development of an autoimmune response of the human body.

The action of xenobiotics can also influence pathobiology of the liver and stimulate local immune responses, since the liver is the main organ responsible for the chemical detoxification of xenobiotics. For example, it was reported that several groups of the patients diagnosed with PBC lived in regions that were located near supertoxic waste storage sites. Although controversial, some epidemiological studies have correlate use of cosmetics and smoking to the development of PBC. Specific environmental factors that may lead to loss of the tolerance to PDC-E2 have been found. They are xenobiotics that can either mimic or modify lipoic acid. These include 2-octynic acid and 6,8-bis-acetylthiooctanoic acid (a metabolite of acetaminophen), which are often used in cosmetics.

Moreover, gamma radiation can also cause autoimmune reactions, since a high number of PBC cases have been found among survivors of the Nagasaki nuclear disaster.

PCB often co-occurs with other autoimmune diseases, including Sjögren's syndrome and chronic thyroiditis.

The importance of genetic factors in the development of PBC has been shown in multiple studies. Studies on the pathogenesis of PBC in twins that had been conducted over the past decade has shown that the incidence rate is higher in monozygotic twins than in dizygotic twins (with a pairwise concordant ratio of 0.63, which is one of the highest ratios for autoimmune diseases), indicating high genetic susceptibility for the development of the disease.

Thus, PBC is now considered a multifactorial polygenic disease, in which the impaired immune tolerance is explained not only by environmental factors, but also by genetic susceptibility and epigenetic phenomena, that greatly contribute to the pathogenesis of the disease. However, the current lack of certainty regarding the causes of the disease makes targeted reduction or elimination of risk factors for PBC, as well as use of targeted preventive measures against its development and effective treatment, impossible.

The course of the PBC development can be divided into the following stages:
Stage 1—a trigger that sets off liver cells damage.
Stage 2—the development of cholestasis and induction of inflammation.
Stage 3—the development of high intensity inflammation.
Stage 4—fibrosis.
Stage 5—cirrhosis.
   1) A Trigger that Sets Off Liver Cells Damage. For PBC, this is an Autoimmune Process.

Damage to the liver cells onsets with an autoimmune response of the body to mitochondrial and nuclear antigens. The most common autoimmune reaction is the synthesis of antimitochondrial antibodies (AMA). AMAs have specificity against members of the family of multienzymatic 2-oxo-acid dehydrogenase (2-OADC) complexes. These complexes, that play an important role in the energetics of mitochondria, have a common multicomponent multidomain structure.

### 2) Development of Cholestasis and Induction of Inflammation.

Cholestasis (cholestatic syndrome) is an impairment of the processes of the bile synthesis and/or bile excretion, resulting in a decrease in the amount of the bile that enters the duodenum. Cholestasis can be asymptomatic or manifest as fatigue, itching in the right upper abdomen, often as so-called cholestatic/obstructive jaundice.

### 3) the Development of High Intensity Inflammation.

Inflammation is an important mechanism for the PBC progression. A high concentration of toxic lipids, mainly free fatty acids (FA), causes cellular stress and induces specific signals that trigger hepatocyte apoptosis, which is the main mechanism of the cell death in PBC, and correlates with the degree of liver inflammation and fibrosis. Various types of the human immune cells, such as monocytes, macrophages, and others, also migrate to the site of inflammation contributing to the development and progression of PBC.

### 4) Fibrosis.

Chronic inflammation and damage to the liver cells leads to the development of the fibrotic processes, as a result of which the connective tissue replaces the normal liver parenchymal tissue and forms scars. Growth factors, cytokines and chemokines secreted by activated macrophages play a central role in the activation of fibrosis. Connective tissue is formed by the production of a huge amount of extracellular matrix proteins (mainly collagen I and III) caused by monocyte-derived macrophages, resident macrophages, as well as damaged hepatocytes, hepatic stellate cells (HSC) and some other cell types. This results in the replacement of the normal liver tissues with the formation of the scar tissue and circulatory disorders inside the organ. The development of liver fibrosis leads to significant changes in the quantity and quality of the hepatic extracellular matrix, impaired liver detoxification function, liver failure and, at the last stage, liver cirrhosis.

### 5) Cirrhosis.

Cirrhosis occurs when the liver undergoes significant damage as a result of the fibrosis progression. The scar tissue gradually replaces the normal liver tissue, blocks portal blood flow (resulting in increased pressure in the portal vein and portal hypertension development) and leads to the inability of the liver to perform its functions. Liver cirrhosis progresses over time with manifestation of the decompensation symptoms. The mean survival rate of the patients with liver cirrhosis after the first 5 years from the onset of the first symptoms of decompensation is 45%, after 10 years-only 10-20%. The main treatment for decompensated cirrhosis is liver transplantation.

In rare cases, PBC is complicated by hepatocellular carcinoma-HCC (approximately 2-2.5% of all cases of PBC incidence). The results of epidemiological studies of the correlation between PBC and HCC are generally consistent with the idea that liver cirrhosis is a risk factor for the development of HCC. It is not currently possible to determine whether there are any other PBC-specific risk factors for the development of HCC other than cirrhosis.

Primary biliary cholangitis has no specific symptoms, therefore it is very difficult to be detected and diagnose correctly in its early stages. Usually the patients experience tiredness (up to 80% of cases) and depression. In some cases, hypothyroidism, anemia, obesity, dry skin and eyes manifest. Itching, usually mild to moderate in intensity, occurs in 20-70% of the patients. Itching correlates with general fatigue and negatively affects night sleep, which can significantly reduce the patient's quality of life.

In later stages of the disease, the following symptoms may develop:
nausea;
abdominal pain;
loss of appetite;
weight loss;
joint pain;
jaundice;
xanthoma and/or xanthelasma;
accumulation of ascites in the abdominal cavity;
systemic edema.

Currently, due to non-specific symptoms, or asymptomatic course of the disease, PBC in the early stages is most often diagnosed incidentally during a general physical examination. In people with suspected PBC, the diagnosis is based on biochemical methods, such as determination of the levels of ALT (alanine aminotransferase) and AST (aspartate aminotransferase) liver enzymes, as well as total bilirubin, antimitochondrial (AMA) and antinuclear (ANA) antibodies in the blood serum. Sometimes additional non-invasive research methods (ultrasound, MRI, etc.) are performed. In rare cases, a biopsy may be taken. A high titer of AMA is observed in more than 95% of the patients with PBC.

However, determining levels of these markers is not enough to establish an accurate clinical picture for the patient with PBC, because they are superficial and do not reflect the processes occurring in the liver at the cellular and molecular level. Levels of AMA/ANA markers indicate the presence or absence of the autoimmune processes. The level of liver enzymes enables establishing only the fact of the liver cell destruction. Generally, said markers enable determining only the stage of cirrhosis and the general condition of the liver. Thus, based on the imperfect results of the study, it is impossible for the physician to determine the stage of PBC and administer an effective treatment to cease its development, especially in the early stages of PBC (stages 1-2).

PBC is a chronic, progressive disease. Without appropriate treatment, PBC leads to chronic inflammation, fibrosis, and eventually cirrhosis of the liver. There is no specific treatment for PBC, and current therapies are aimed at arresting the progression of the disease and relieving symptoms. Essentially, an effective treatment or an adequate patient response to the treatment means that a suppression in the PBC progression has been achieved. Current protocols for the PBC treatment include solid dosage forms comprising ursodeoxycholic acid (UDCA) and solid dosage forms comprising obeticholic acid (OCA).

For example, there is a known immediate release solid dosage form called URSO 250® for the treatment of primary biliary cholangitis, comprising a core comprising ursodeoxycholic acid and one layer comprising film coating. The dosage form is further specified as tablet for oral administration comprising 250 mg of ursodeoxycholic acid per dosage form, microcrystalline cellulose, povidone, sodium starch glycolate, magnesium stearate, ethylcellulose, dibutyl sebacate, carnauba wax, hydroxypropyl methylcellulose, PEG 3350, PEG 8000, cetyl alcohol, sodium lauryl sulfate and hydrogen peroxide. (https://www.accessdata.fda.gov/drugsatfda_docs/label/2008/020675s0131bl.pdf). The allocation of the ingredients between the core and the layer of the dosage form is not specified. UDCA, and thus URSO 250®, is the first-line solid dosage form administered for all patients with diagnosed PBC, regardless of the disease stage, with exception of decompensated cirrhosis (one of the few contraindications for UDCA use). The recommended daily dose of the known solid dosage form is 13-15 mg of UDCA per 1 kg of the patient body weight, divided into 2 or 4 intakes with food (4-5 tablets per day based on 80 kg of the patient body weight). The daily dose may be adjusted by the physician according to the patient condition.

The first stage of PBC treatment continues for 1 year, after which the patient biochemical response to UDCA is determined and the effectiveness of the treatment is evaluated. The effectiveness is evaluated using various evaluation systems, such as Paris, Paris2, Rotterdam, etc. In particular, according to the Paris2 system, the criteria for the satisfactory treatment of PBC are:
ALT level ≤3-fold upper limit of normal (ULN);
AST level ≤2-fold ULN;
normal bilirubin level.

The effectiveness of the PBC treatment depends on many factors, the main being the stage of the disease development. Patient age and gender have also been shown to influence response and outcomes of the PBC treatment. Younger patients (under 45 years) tend to have symptomatic PBC and are less likely to respond to standard UDCA therapy. As a result, this population has a higher standardized mortality rate, in particular, there are more deaths due to liver disorders, while older people are more likely to die from concomitant non-liver causes. Male sex is associated with a delayed time of diagnosis, a poorer biochemical response to UDCA therapy, and a greater risk of developing hepatocellular carcinoma.

According to statistics, only for 60% of the patients the treatment of PBC with solid dosage forms comprising UDCA is successful (i.e., there is an arresting of the PBC development). The other 40% of the patients with an inadequate response to UDCA treatment (when PBC continues to progress, and the patient condition worsens) are treated with solid dosage forms comprising OCA.

Thus, there is a known immediate release solid dosage form for the treatment of primary biliary cholangitis called OCALIVA®, comprising a core comprising obeticholic acid and one layer comprising film coating. The dosage form is further specified as a tablet for oral administration comprising in the core obeticholic acid (5 or 10 mg per dosage form), microcrystalline cellulose, sodium starch glycolate, magnesium stearate and comprising in the layer a non-enteric coating Opadry II (Yellow) comprising polyvinyl alcohol-part hydrolyzed, titanium dioxide, macrogol (polyethylene glycol 3350), talc, iron oxide yellow. (https://www.accessdata.fda.gov/drugsatfda_docs/label/2018/207999s0031bl.pdf). Before starting OCALIVA® in the patients with suspected liver cirrhosis, a nomogram should be used to determine the cirrhosis stage according to the Child-Pugh system (A, B or C) and establish the appropriate initial dose of the API. The recommended starting dose and titration regimen for OCALIVA® in the patients who have not achieved an adequate biochemical response to an appropriate dose of UDCA (high-risk group) for at least 1 year, or who are intolerant to UDCA, depends on the disease stage.

For the patients, who have not yet developed cirrhosis or with type A compensated liver cirrhosis, OCALIVA® is administered for the first 3 months in an amount corresponding to 5 mg of OCA per day. If after 3 months the patient has not achieved an adequate decrease in alkaline phosphatase (ALP) and/or total bilirubin, the daily dose is increased to 10 mg of OCA (providing that the patient tolerates it well). The maximum daily dose of OCALIVA® for this category of the patients is 10 mg of OCA. Before adjusting the dose, it is recommended to recalculate the dose according to the Child-Pugh system.

For the patients with type B or C compensated liver cirrhosis or with a previous case of decompensation (gastroesophageal variceal bleeding, new case or worsening of jaundice, spontaneous bacterial peritonitis, etc.), during the first 3 months, OCALIVA® is administered in an amount corresponding to 5 mg of OCA per week. If after 3 months the patient has not achieved an adequate reduction in alkaline phosphatase and/or total bilirubin, the dose is increased to 5 mg of OCA twice a week, with an interval between the doses of at least 3 days, with a possible titration to 10 mg of OCA twice a week, with an interval between the doses of at least 3 days. The dosage depends on the response and condition of the patient. The maximum dose of OCALIVA® for this category of the patients is 10 mg of OCA per week, with an interval between the doses of at least 3 days. Before adjusting the dose, it is recommended to recalculate the dose according to the Child-Pugh system.

Administration of the known URSO 250® and OCALIVA® solid dosage forms in compliance with the current protocols for the PBC treatment is associated with a large number of negative consequences.

The peculiarity of PBC is that the initial stages of PBC are almost never identified. The use of the current research protocols and markers enables diagnosing PBC only in the later stage when the disease progresses over time and begins to manifest itself.

In addition, the diagnosis of PBC is based on the determination of the levels of the outdated markers (ALT, AST, bilirubin), which is not an effective approach, since it does not allow identifying the stage of PBC. The lack of accurate information about the patient diagnosis, the degree of disease progression and the patient condition does not allow the physician to administer an adequate and effective treatment regimen using the known solid dosage forms URSO 250® and OCALIVA®. As a result, a year is spent trying to use solid dosage forms with UDCA (in particular, URSO 250®) and determine the stage of PBC.

Moreover, starting from the second stage of PBC, it is no longer advisable to waste time treating the patient with the UDCA solid dosage forms, since the percentage of the development and transition to irreversible stages of the disease is high. Therefore, an accurate and timely diagnosis of the PBC stage will result in the physician being able to prescribe safer and more effective treatment.

The therapeutic factor is the main disadvantage. OCALIVA® solid dosage form received fast-track USFDA approval for the treatment of PBC, confirming the great need for new solid dosage forms and methods of PBC treatment. In particular, such a need remains very urgent given the large percentage (40%) of the patients with an inadequate response to the treatment with solid dosage forms comprising UDCA, such as URSO 250®. However, the current protocols for the PBC treatment involve using solid dosage forms comprising OCA (particularly, OCALIVA®) only after the end of the first stage of the treatment, which continues for 12 months and includes the use of solid dosage forms comprising UDCA. Over such a period of time, the patients with an inadequate response to UDCA treatment achieve no therapeutic effect. PBC not only does not stop its development, but also continues to progress, transit to more serious stages, that are much more dangerous for the patients and significantly worsen their quality of life. At the last stages of the PBC development, cirrhosis manifests itself, which significantly reduces the survival rate of the patients, even after liver transplantation.

The second drawback is compliance. The patient compliance with all physician recommendations on the treatment regimen is a very important factor for the efficient treatment of chronic diseases such as PBC. To achieve positive therapeutic results, the patient must follow many rules, such as taking medicines on time and in the required dose, following the physician recommendations regarding lifestyle and nutrition, and maintaining a positive psychological (emotional) condition. The patient propensity for the treatment, or compliance, depends on many factors:
age, emotional condition, general level of education;
the number of solid dosage forms to be taken and the frequency of their intake per day;
characteristics of the solid dosage form that make administration inconvenient or uncomfortable (size, taste, smell, etc.);
the timing and predictability of the perceived benefit from therapy;
insufficient information provided to the patient regarding the condition and the prescribed regimen;
limitations imposed on the patient's usual daily activities;
side effects associated with the intake of solid dosage forms;
the financial burden associated with obtaining the prescribed solid dosage forms.

Considering the current situation regarding the PBC treatment protocols, it can be concluded that patients suffering from PBC are characterized by low compliance, thus, known solid dosage forms for the treatment of PBC, such as URSO 250® and OCALIVA®, also have poor compliance. This is due to several reasons.

First, adherence to the treatment regimen may be difficult for the elderly, who find it troublesome to keep track of the number of the doses and dosing of the drug.

Secondly, the first stage of the PBC therapy involves long-term daily use of the drugs with high doses of UDCA (up to 4 doses per day). At the same time, the therapeutic effect of such drugs, for example, URSO 250®, does not appear quickly, but along with the accumulation of the active pharmaceutical ingredient (API) in the patient body. Given the nature of the disease and current methods of its treatment, the positive results of the PBC treatment can often be determined only by a medical expert. Moreover, in 40% of cases of using the drugs comprising UDCA, an adequate response to the treatment is not observed at all, and the patient condition worsens even more. This significantly undermines the patient confidence in the physician, the therapy regimen and drugs administered, and reduces the incentive for further treatment.

Another disadvantage of such long-term use of the drugs is the change in the regimen in accordance with the disease stages. The PBC treatment is a process extended over years. At the same time, the emotional condition of the patients is rather unstable, they experience constant fatigue and irregularity of behavior, therefore, when the patient gets used to particular algorithm for taking the drugs, it is desirable for them to keep this algorithm during further treatment. Today, the treatment is accomplished with several drugs that alternate in time, while the drug which regimen does not need to be changed (only the dose changes) will have a much greater compliance.

Moreover, a disadvantage is the economic factor. During the first stage of the PBC treatment, which continues for 12 months, the patient spends money on the UDCA drugs, although, after completion of the first stage of the PBC treatment, almost in half of the patients the UDCA drugs are recognized to be ineffective.

Considering the above reasons, there is a high probability that the patient suffering from PBC will not appropriately follow the physician recommendations for the treatment of PBC, as a result of which the effectiveness of the disease treatment will be significantly less.

Thus, the current situation is as follows: the molecular mechanisms of the PBC development are not studied, the disease stages are not diagnosable, PBC is almost never diagnosed in the early stages, therefore, the PBC therapy is now carried out simply upon identification of the disease or detection of liver cirrhosis. Whatever stage of PBC the patient has at the start of the treatment, they are treated first with the UDCA drugs for a year, only then consideration is given to the OCA drugs at a dose of either 5 mg/day, with a possible dosage increase to 10 mg/day, or, in the presence of decompensated cirrhosis of B and C stages, 5 mg or 10 mg, OCA 1-2 times per week. During the first year of the treatment with the UDCA drugs, which is spent on diagnosing and dividing patients into low-risk and high-risk groups, in 40% of the patients, the PBC progression not only does not stop, but continues, and transits to more severe and irreversible stages of the development. Thus, the first year is no less significant than the process of diagnosing the disease as a whole and should be spent not on clarifying the diagnosis and selecting treatment, but directly on the adequate treatment. Current protocols of PBC treatment do not provide opportunities to administer effective treatment from the first visit to the physician.

Additionally, currently, there is no drug for the treatment of all stages of PBC.

Thus, now there is a need for a global change in the principles and approaches to the diagnosis and treatment of PBC.

### SUMMARY OF THE INVENTION

The objective technical problems to be solved are:
development of a new drug that would increase the effectiveness of the PBC treatment from 60% to more than 80% with the possibility of being used in all stages of the disease;
development of a new drug with a structure that prevents the competition of two active pharmaceutical ingredients (APIs) for the transporters.

The objective technical problem is solved with a modified release solid dosage form for the treatment of primary biliary cholangitis, comprising:
(a) a core comprising ursodeoxycholic acid; and
(b) at least one layer comprising obeticholic acid,
wherein a weight ratio of ursodeoxycholic acid to obeticholic acid ranges from 2:1 to 1000:1.

In one aspect, the weight ratio of ursodeoxycholic acid to obeticholic acid ranges from 500:1 to 1000:1.

In another aspect, the weight ratio of ursodeoxycholic acid to obeticholic acid ranges from 700:1 to 1000:1.

In one aspect, the dosage form comprises the core and three layers, wherein the core comprises ursodeoxycholic acid and at least one of the layers comprises obeticholic acid.

In one aspect, the dosage form comprises an outer layer, a second layer comprising obeticholic acid, a third layer, and the core comprising ursodeoxycholic acid.

In one aspect, the outer layer comprises an enteric coating or a layer resistant to gastric acid.

In another aspect, the outer layer comprises a non-enteric coating or a gastric acid soluble layer.

In one aspect, the second layer comprises obeticholic acid in an amount of 1-50 mg. In one aspect, the third layer comprises a STOP layer with a delayed dissolution or an enteric coating.

In one aspect, the dissolution time of the STOP layer is 1-2 hours.

In one aspect, the core comprises ursodeoxycholic acid in an amount of 100-1000 mg. In one aspect, the core provides the modified release of ursodeoxycholic acid over a period of 1 to 6 hours.

In one aspect, the solid dosage form comprises:
(a) the outer layer comprising the enteric coating;
(b) the second layer comprising obeticholic acid;
(c) the third layer comprising the STOP layer; and
(d) the core comprising ursodeoxycholic acid.

In another aspect, the solid dosage form comprises:
(a) the outer layer comprising the gastric acid soluble layer;
(b) the second layer comprising obeticholic acid;
(c) the third layer comprising the enteric coating; and
(d) the core comprising ursodeoxycholic acid.

In one aspect, the dosage form is for the treatment of primary biliary cholangitis characterised by the following concentration level values of markers IL-6, Nf-kB, MCP-1/CCL2, and Bcl-2 in a patient sample:

| | |
|---|---|
| IL-6 | at least 3.0 pg/ml; |
| Nf-kB | at least 14.0 pg/ml; |
| MCP-1/CCL2 | at least 215 pg/ml; |
| Bcl-2 | at least 0.24 U/ml. |

In one aspect, primary biliary cholangitis is characterised by the following concentration level values of markers IL-6, Nf-kB, MCP-1/CCL2, Bcl-2, TNF-α, MDA in a patient sample:

| | |
|---|---|
| IL-6 | at least 3.0 pg/ml; |
| Nf-kB | at least 14.0 pg/ml; |
| MCP-1/CCL2 | at least 215 pg/ml; |
| Bcl-2 | at least 0.24 U/ml; |
| TNF-α | at least 0.14 ng/ml. |
| MDA | at least 4.3 nmol/ml. |

For purposes of this specification, the following terms are defined.

As used herein, the term ‘layer’ denotes a discrete stratum within a solid dosage form. A layer may be formed by compression, granulation, molding, or other formation techniques. A layer may include or exclude an active pharmaceutical ingredient and may serve structural, separation, protective, or release-modifying roles, but is not restricted to any particular function unless expressly specified. The term encompasses external layers and layers located between internal strata.

As used herein, the term ‘coating’ denotes an applied layer formed on a surface of the solid dosage form or on the surface of a component thereof. A coating may be positioned on the exterior of the dosage form or between internal strata. Coatings are formed using application techniques such as spraying, dipping, pan-coating, or fluid-bed coating. The coating may be functional or non-functional and includes film coatings, polymer coatings, barrier coatings, and sealing coats unless otherwise indicated.

As used herein, the term ‘layer resistant to gastric acid’ refers to any layer, applied or otherwise, within a solid dosage form that substantially resists dissolution or degradation under acidic conditions. Such a layer may be external or internal, functional or non-functional, and includes coatings or compressed strata unless otherwise indicated.

As used herein, the term ‘enteric coating’ denotes a coating applied to a solid dosage form or a component thereof that resists dissolution under acidic conditions (e.g., gastric fluid at pH˜1-3) and releases its contents at higher pH (e.g., intestinal fluid at pH˜5-8). An enteric coating is an applied layer and thus is a subset of ‘coatings’ as defined herein.

As used herein, the term ‘gastric acid soluble layer’ refers to any layer, applied or otherwise, within a solid dosage form that substantially dissolves or disintegrates under acidic conditions. Such a layer may be external or internal, functional or non-functional, and includes coatings or compressed strata unless otherwise indicated.

As used herein, the term ‘non-enteric coating’ denotes a coating applied to a solid dosage form or a component thereof that is not designed to resist dissolution in acidic conditions. A non-enteric coating may dissolve or disintegrate in gastric pH and may be positioned on the exterior or between internal strata. A non-enteric coating is an applied layer and thus is a subset of ‘coatings’ as defined herein.

As used herein, the term ‘outer layer’ denotes a layer positioned on the external surface of a solid dosage form. An outer layer may be formed by compression, coating, or other techniques, and may serve protective, aesthetic, or release-modifying purposes. The outer layer is a subset of ‘layers’ as defined herein.

As used herein, the term ‘STOP layer’ denotes a discrete layer within a solid dosage form that separates adjacent layers to prevent interaction and modulate the temporal release of APIs. A STOP layer may be applied or formed by compression, may be external or internal, and is a subset of ‘layers’ as defined herein.

Since the mechanism of the PBC onset is currently unknown and it is impossible to clearly diagnose PBC with the definition of the disease stage, the current treatment protocols involve the use of the UDCA drugs, that promote relief of the disease syndromes by removing bile acids (BA). However, studies conducted by the authors using the additional group of markers to determine the processes of inflammation and identify apoptosis and fibrosis in PBC, show that starting from the second stage of PBC, UDCA is no longer effective. At the later stages of the disease, in addition to stimulating the processes of elimination of fatty acids, it also becomes necessary to stop the FA synthesis. Thus, in the absence of the studies on the molecular mechanism and diagnosis of PBC, it was not possible and appropriate to use the solid dosage form that comprises both acids, UDCA and OCA.

Since compliance is especially important for the PBC patients (as described above), this objective is achieved by developing a solid dosage form for the PBC treatment that comprises UDCA and OCA.

Having conducted their own research, both theoretical and experimental, the authors of the present disclosure concluded that the state-of-the-art system for diagnosing and treating PBC has many disadvantages and needs to be modified.

Currently, for the PBC treatment, the drugs comprising UDCA are used first, and if there is no efficacy or there is intolerance, the drugs with OCA are administered. In order to better understand the disadvantages of this approach, it is necessary to consider the mechanisms of action of both acids.

Experimental data indicate three main mechanisms of UDCA action:
protection of cholangiocytes against cytotoxic hydrophobic bile acids;
stimulation of hepatobiliary secretion;
protection of hepatocytes against apoptosis caused by bile acids.

The protective function of UDCA against cholestasis is achieved, in particular, by the utilization of accumulated bile acids. However, the secretory capacity of hepatocytes is closely related to the presence of transport proteins in the canalicular membrane. The action of UDCA is to stimulate the expression of proteins of bile salt exporter pumps (BSEP), proteins associated with resistance to various drugs (MDR3) and (MRP4), and also promotes the elimination of bile acids from hepatocytes.

In addition, another mechanism of hepatocyte protection against toxic bile acids is the inhibition of BA uptake by intestinal lumen transporters due to UDCA competition for transporter binding sites.

Clinical studies on UDCA efficacy have demonstrated a decrease in the level of aminotransferases, alkaline phosphatase and bilirubin in the patient's blood serum, regardless of the improvement of processes at the histological level. There remains debate regarding the efficacy of UDCA in terms of overall patient survival, the impact on the need for liver transplantation or achieving improvements at the histological level.

The debate on whether UDCA affects survival and mortality in patients without liver transplantation is covered in review articles and meta-analyses, where scientists agree on a certain control of biochemical parameters in the patients taking UDCA, while the effect on histopathology and survival without transplantation cannot be assessed in studies. However, the fact is that UDCA is not an all-inclusive drug for the treatment of PBC and does not always stop disease progression.

Furthermore, asymptomatic PBC cases have a favorable natural history and should not be considered in the same cohort as symptomatic PBC. Of particular note, two out of three asymptomatic PBC patients benefit from UDCA use compared to those with an equal or better natural history, with 10-year survival rates of 57%, 70%, and over 90% reported in untreated patients. Additionally, the expected median survival of asymptomatic PBC patients was 10 and 16 years in two large cohorts followed for 24 years, while the median survival of symptomatic patients is approximately 7 years.

A systematic review of 16 randomized clinical trials evaluating the efficacy of UDCA in PBC compared with placebo did not demonstrate a significant effect of UDCA on mortality or the need for liver transplantation in patients. The effects of UDCA do not extend beyond a certain improvement in serum bilirubin levels, liver function tests for ALT and AST, and alkaline phosphatase. It was found that UDCA does not improve the course of symptom manifestation, such as pruritus and fatigue, autoimmune conditions, liver histology, and portal pressure. In addition, the use of UDCA was associated to great extend with the development of adverse events such as weight gain.

Considering these data, it can be concluded that although UDCA has some therapeutic activity and proven efficacy, it is effective only in the early stages of PBC and in asymptomatic patients.

### Mechanism of Action of Obeticholic Acid

OCA is a derivative of the human primary bile acid, chenodeoxycholic acid, and a first-in-class agonist that selectively binds to the nuclear farnesoid X receptor FXR. Due to chemical modification, OCA has a 100-fold higher affinity for FXR than chenodeoxycholic acid, a natural FXR agonist. FXR is one of the key transcription factors that is overexpressed in the liver, kidneys, intestines, and adrenal glands, and plays a key role in the pathogenesis of inflammatory liver diseases. FXR has anti-inflammatory properties due to the inhibition of the NF-κB gene. FXR regulates bile acid synthesis through two different mechanisms. In the liver, FXR increases the expression of the small heterodimer partner (SHP) gene. The SHP gene suppresses the transcription of bile acid synthesis enzymes such as CYP7A1 and CYP8B1, thereby reducing bile acid synthesis in hepatocytes. In addition, FXR regulates the activity of bile acid transport systems in hepatocytes. In hepatocytes, FXR is involved in liver inflammation, fibrosis, regulation of metabolic pathways, and regeneration.

In addition to its central role in bile acid metabolism, FXR activation also regulates the expression of various genes involved in glucose, lipid, and lipoprotein metabolism. Hepatic FXR suppresses fatty acid synthesis and uptake and enhances beta-oxidation, regulating lipid homeostasis.

OCA suppresses metabolic stress-induced p53 activation and apoptosis of damaged hepatocytes. Moreover, in addition to hepatocytes, OCA has been shown to exhibit anti-inflammatory and antifibrotic properties in immune cells, vascular smooth muscle cells, endothelial cells, and fibroblasts in vitro and in vivo.

It has also been shown that FXR stimulation in hepatic macrophage cells with OCA exerted anti-inflammatory effects, inhibited fibrosis, and stimulated liver tissue regeneration.

### Comparison of the Actions of UDCA and OCA

As was said above, the main mechanisms of action of UDCA are immunomodulatory action, inhibition of absorption of hydrophobic FA from the intestinal lumen and stimulation of FA release from hepatocytes, while for OCA this is modulation of FXR action, anti-inflammatory and antifibrotic action, inhibition of FA synthesis.

Based on the above 5 stages of PBC development (1—trigger that starts liver cell damage; 2—development of cholestasis and induction of inflammation; 3—development of high-intensity inflammation; 4—fibrosis; 5—cirrhosis), UDCA functions only in the first two stages, since its anti-inflammatory properties are extremely weak, and OCA starts functioning from the second to the last stage. From the second stage of PBC, it is no longer advisable to waste time on treating the patient with drugs comprising UDCA, since the risk of disease development and transition to irreversible stages is high. Therefore, accurate and timely diagnosis of the PBC stage will lead to the fact that it is possible to administer a safer and more effective treatment and use a mixture of two acids. Since the compliance is especially important for the patients with PBC, the objective is solved by developing a solid dosage form for the treatment of PBC, comprising both UDCA and OCA.

### EMBODIMENTS OF THE INVENTION

To date, there is no drug effective for the treatment of PBC at all stages of its progression.

As a result of the studies on the use of the markers in the treatment of PBC, it was shown that patients diagnosed with PBC have an increase in the markers of inflammation and apoptosis, which indicates the advisability of changing the protocols for the treatment of PBC, in particular, the need for the simultaneous use of UDCA and OCA in one dosage form.

To this end, a new all-purpose solid dosage form for the treatment of PBC, that comprises both ursodeoxycholic and obeticholic acids, and is effective for the use at all stages of PBC due to its complex mechanism of action, was developed. In particular, the use of the new solid dosage form allows simultaneous blockage of the transport and synthesis of bile acids and allows achieving a pronounced hepatoprotective effect.

To investigate the feasibility of producing solid dosage forms comprising ursodeoxycholic acid and obeticholic acid for the treatment of PBC, such dosage forms were prepared and evaluated.

### Example 1

Table 1 presents exemplary implementations of solid dosage forms comprising ursodeoxycholic acid and obeticholic acid. In each case, the solid dosage forms were tablets configured for immediate release of both APIs, reflecting a typical approach in solid dosage form design.

TABLE 1
Exemplary formulations of solid dosage forms in tablet form

| Embodiment No. | Description | Composition |
|---|---|---|
| Embodiment | Coated tablet | Ursodeoxycholic acid-100 mg |
| 1 | | Obeticholic acid-1 mg |
| | | Excipient |
| Embodiment | Uncoated tablet | Ursodeoxycholic acid-100 mg |
| II | | Obeticholic acid-2.5 mg |
| | | Excipient |
| Embodiment | Capsule | Ursodeoxycholic acid-100 mg |
| III | | Obeticholic acid-5 mg |
| | | Excipient |
| Embodiment | Coated tablet | Ursodeoxycholic acid-100 mg |
| IV | | Obeticholic acid-10 mg |
| | | Excipient |
| Embodiment | Uncoated tablet | Ursodeoxycholic acid-100 mg |
| V | | Obeticholic acid-20 mg |
| | | Excipient |
| Embodiment | Capsule | Ursodeoxycholic acid-100 mg |
| VI | | Obeticholic acid-50 mg |
| | | Excipient |
| Embodiment | Coated tablet | Ursodeoxycholic acid-250 mg |
| VII | | Obeticholic acid-1 mg |
| | | Excipient |
| Embodiment | Uncoated tablet | Ursodeoxycholic acid-250 mg |
| VIII | | Obeticholic acid-2.5 mg |
| | | Excipient |
| Embodiment | Capsule | Ursodeoxycholic acid 250 mg |
| IX | | Obeticholic acid-5 mg |
| | | Excipient |
| Embodiment | Coated tablet | Ursodeoxycholic acid-250 mg |
| X | | Obeticholic acid-10 mg |
| | | Excipient |
| Embodiment | Uncoated tablet | Ursodeoxycholic acid-250 mg |
| XI | | Obeticholic acid-20 mg |
| | | Excipient |
| Embodiment | Capsule | Ursodeoxycholic acid-250 mg |
| XII | | Obeticholic acid-50 mg |
| | | Excipient |
| Embodiment | Coated tablet | Ursodeoxycholic acid-500 mg |
| XIII | | Obeticholic acid-1 mg |
| | | Excipient |
| Embodiment | Uncoated tablet | Ursodeoxycholic acid-500 mg |
| XIV | | Obeticholic acid-2.5 mg |
| | | Excipient |
| Embodiment | Capsule | Ursodeoxycholic acid-500 mg |
| XV | | Obeticholic acid-5 mg |
| | | Excipient |
| Embodiment | Coated tablet | Ursodeoxycholic acid-500 mg |
| XVI | | Obeticholic acid-10 mg |
| | | Excipient |
| Embodiment | Uncoated tablet | Ursodeoxycholic acid-500 mg |
| XVII | | Obeticholic acid-20 mg |
| | | Excipient |
| Embodiment | Capsule | Ursodeoxycholic acid-500 mg |
| XVIII | | Obeticholic acid-50 mg |
| | | Excipient |
| Embodiment | Coated tablet | Ursodeoxycholic acid-1000 mg |
| XIX | | Obeticholic acid-1 mg |
| | | Excipient |
| Embodiment | Uncoated tablet | Ursodeoxycholic acid-1000 mg |
| XX | | Obeticholic acid-2.5 mg |
| | | Excipient |
| Embodiment | Capsule | Ursodeoxycholic acid-1000 mg |
| XXI | | Obeticholic acid-5 mg |
| | | Excipient |
| Embodiment | Coated tablet | Ursodeoxycholic acid-1000 mg |
| XXII | | Obeticholic acid-10 mg |
| | | Excipient |
| Embodiment | Uncoated tablet | Ursodeoxycholic acid-1000 mg |
| XXIII | | Obeticholic acid-20 mg |
| | | Excipient |
| Embodiment | Capsule | Ursodeoxycholic acid-1000 mg |
| XXIV | | Obeticholic acid-50 mg |
| | | Excipient |

The solid dosage forms listed in Table 1 can be prepared by various methods, some of which are described below. The methods are carried out in accordance with technical instructions for manufacturing solid dosage forms, including the components and their quantities, the equipment used, and the conditions and duration of each stage.

### Method 1

Step 1: Weighing. Weigh the required amount of the active pharmaceutical ingredients and excipients of the solid dosage form.

Step 2: Sifting of the raw materials. Sift the active pharmaceutical ingredients and excipients through a sieve with a specific mesh size.

Step 3: Dry mixing. Transfer the sifted components to a dry mixing apparatus and mix for a specific period of time at a specific mixing speed to produce a dry mixture.

Step 4: Preparing a slurry. Mix a specific volume of the purified water EP (according to European Pharmacopoeia) having a specific temperature with a base to produce a slurry to prepare a slurry.

Step 5: Granulating. Transfer the slurry prepared in Step 4 to a granulator and mix the slurry with the dry mixture produced in Step 3 for a specific period of time at a specific mixing speed, to prepare granules.

Step 6: Semi-drying. Transfer the granules produced in Step 5 to the drying apparatus and dry at a specific temperature for a specific period of time, to produce dried granules.

Step 7: Crushing and sifting. Sift the dried granules from Step 6 through a sieve with a certain mesh size. Ground the granules of a larger size in an all-purpose grinder and pass through a sieve with a certain mesh size.

Step 8: Final drying. Transfer the granules produced in Step 7 to the drying apparatus and dry at a specific temperature for a specific period of time.

Step 9: Lubricating. Transfer the granules produced in Step 8 to a blender and mix for a specific period of time at a specific mixing speed. Add a material to lubricate the granules and, if necessary, other active pharmaceutical ingredients and/or excipients, and mix for a specific period of time at a specific mixing speed to prepare lubricated granules.

Step 10: Compressing tablets. Compress the lubricated granules prepared in Step 9 into the solid dosage forms of the tablets of a specified weight and size.

Step 11: Tablet coating. Coat the tablets with coating.

### Method 2

Step 1: Weighing. Weigh the required amount of the active pharmaceutical ingredients and excipients of the solid dosage form.

Step 2: Sifting of the raw materials. Sift the active pharmaceutical ingredients and excipients through a sieve with a specific mesh size.

Step 3: Dry mixing. Transfer the sifted components to a dry mixing apparatus and mix for a specific period of time at a specific mixing speed to produce a dry mixture.

Step 4: Adding granulating solution and wetting. Add pre-prepared granulating solution to the dry mixture produced in Step 3 and mix for a specific period of time at a specific mixing speed.

Step 5: Granulating. Transfer the mixture produced in Step 4 to the granulator and mix for a specific period of time at a specific mixing speed to produce granules.

Step 6: Drying. Transfer the granules produced in Step 5 to the drying apparatus and dry at a specific temperature for a specific period of time.

Step 7: Granule coating. Coat the granules with a coating.

### Method 3

Step 1: Weighing. Weigh the required amount of the active pharmaceutical ingredients and excipients of the solid dosage form.

Step 2: Sifting of the raw materials. Sift the active pharmaceutical ingredients through a sieve with a specific mesh size.

Step 3: Dry mixing. Transfer the sifted components to a dry mixing apparatus and mix for a specific period of time at a specific mixing speed to produce a dry mixture.

Step 4: Preparing of the granulating solution. Add the granulating solution base and purified water EP into the preparation container and mix for a specific period of time to produce a granulating solution.

Step 5: Adding of the granulating solution and wetting. To the dry mixture produced in Step 3, add the granulating solution produced in Step 4 and mix for a specific period of time at a specific mixing speed.

Step 6: Granulating. Transfer the mixture produced in Step 5 to the granulator and mix for a specific period of time at a specific mixing speed to produce granules.

Step 7: Drying. Transfer the granules produced in Step 6 to the drying apparatus and dry at a specific temperature for a specific period of time.

Step 8: Granule coating. Coat the granules with a coting.

### Method 4

Step 1: Weighing. Weigh the required amount of the active pharmaceutical ingredients and excipients of the solid dosage form.

Step 2: Sifting of the raw materials. Sift the active pharmaceutical ingredients through a sieve with a specific mesh size.

Step 3: Dry mixing. Transfer the sifted components to a dry mixing apparatus and mix for a specific period of time at a specific mixing to produce a dry mixture.

Step 4: Compressing tablets. Compress the dry mixture prepared in Step 3 into the solid dosage forms (tablets) having the predetermined weight and dimensions.

Step 5: Tablet coating. Coat the tablets with coating.

As an excipient the claimed solid dosage form can comprise any excipients acceptable for use in pharmaceutical industry, in particular, excipients that are used to produce solid dosage forms, such as fillers, binders, antiadherents, glidants, lubricants, disintegrants, plasticizers, sweeteners, opacifiers, flavorants.

As used herein, the term “filler” denotes inert substances used to create the desired bulk, flow properties, and compression characteristics in the preparation of solid dosage forms. Exemplary fillers include, but not limited to, dibasic calcium phosphate, kaolin, lactose, sucrose, mannitol, cellulose and its derivatives, precipitated calcium carbonate, sorbitol, and starch.

As used herein, the term “binder” denotes a substance used to cause adhesion of powder particles in granulations. Exemplary binders include, but not limited to, acacia, tragacanth, alginic acid and salts thereof, gelatin, cellulose and its derivatives, poly(vinylpyrrolidone), liquid glucose, povidone, polyethylene glycol, polypropylene glycol, polyoxyethylene-polypropylene copolymer, polyethylene ester, polyethylene sorbitan ester, polyethylene oxide, guar gum, polysaccharide, bentonites, sugars, invert sugars, compressible sugar, poloxamers, collagen, albumin, and starch and its derivatives.

As used herein, the term “antiadherent” denotes an agent that prevents the sticking of tablet formulation ingredients to punches and dies in a tableting machine during production. Exemplary antiadherents include, but not limited to, magnesium stearate, talc, calcium stearate, glyceryl behenate, polyethylene glycol (PEG), hydrogenated vegetable oil, mineral oil, stearic acid.

As used herein, the term “glidant” denotes an agent used in solid dosage form formulations to promote flowability during the granulation. Exemplary glidants include, but not limited to, colloidal silica, cornstarch, talc, calcium silicate, magnesium silicate, colloidal silicon, silicon hydrogel.

As used herein, the term “lubricant” denotes a substance used to reduce friction during compression or other processing. Exemplary lubricants include, but not limited to, calcium stearate, magnesium stearate, mineral oil, stearic acid, and zinc stearate.

As used herein, the term “disintegrant” denotes a compound used to promote the disruption of the solid mass into smaller particles that are more readily dispersed or dissolved. Exemplary disintegrants include, but not limited to, starches such as corn starch, potato starch, pre-gelatinized and modified starches thereof, clays, such as bentonite, cellulose and its derivatives, such as microcrystalline cellulose, carboxymethylcellulose calcium, cellulose polyacrilin potassium, alginates, sodium starch glycolate, gums such as agar, guar, locust bean, karaya, pectin, tragacanth; crospovidone.

As used herein, the term “plasticizer” denotes compounds capable of plasticizing or softening a polymer or binder used in invention by way of lowering the melting temperature or glass transition temperature of the polymer or binder. Exemplary plasticizers include, but not limited to, low molecular weight polymers, oligomers, copolymers, oils, low molecular weight polyols, multi-block polymers, single block polymers, and glycerin.

As used herein, the term “sweetener” denotes a compound used to impart sweetness. Exemplary sweeteners include, but not limited to, aspartame, dextrose, glycerin, mannitol, saccharin sodium, sorbitol and sucrose.

As used herein, the term “opacifier” denotes a compound used to render a tablet coating opaque. Exemplary opacifiers include, but not limited to, titanium dioxide, talc.

As used herein, the term “flavorant” denotes a compound used to impart a pleasant flavor and often odor. Exemplary flavorants include, but not limited to, synthetic flavor oils and flavoring aromatics and/or natural oils, extracts from plants, leaves, flowers, fruits and combinations thereof, and other substances known to one skilled in the art.

### Method A

Step 1. Weighing. Weigh the required amount of the active pharmaceutical ingredients and excipients (per 100 kg of the total mixture).

1) UDCA+OCA—80 kg.

2) Corn starch—15 kg.

3) Lactose—3 kg.

4) Povidone—2 kg.

Step 2. Sifting of the raw materials. Sift the components through a sieve with a mesh size of at least 30 mesh.

Step 3. Dry mixing. Transfer the sieved components to a dry mixer and mix them for 1 hour to prepare a dry mixture.

Step 4. Compressing. Transfer the dry mixture produced in Step 3 to a compression machine, and compress into the solid dosage forms (tablets).

Step 5. Tablet coating. Transfer the tablets produced in Step 4 to a tablet film coating machine. Coat the tablets with the film by immersion in an ethanol solution of a film former (eudragit E).

Step 6. Drying. Dry the film-coated tablets prepared in Step 5 to produce the finished solid dosage forms of the film-coated tablets.

### Method B

Step 1. Weighing. Weigh the required amount of the active pharmaceutical ingredients and excipients (per 150 kg of the total mixture).

1) UDCA+OCA—112.5 kg.

2) Corn starch—16.5 kg.

3) Lactose—18.0 kg.

4) Povidone—3.0 kg.

Step 2. Sifting. Sift the components through a sieve with a mesh size of at least 30 mesh. Step 3. Dry mixing. Transfer the sieved components to a dry mixer and mix them for 1 hour to prepare a dry mixture.

Step 4: Compressing. Transfer the dry mixture produced in Step 3 to a compression machine, and compress into the solid dosage forms (tablets).

Step 5. Tablet coating. Transfer the tablets produced in Step 4 to a tablet film coating machine. Coat the tablets with the film by immersion in an ethanol solution of a film former (eudragit L).

Step 6. Drying. Dry the film-coated tablets prepared in Step 5 to produce the finished solid dosage forms of the film-coated tablets.

### Method C

Step 1. Weighing. Weigh the required amount of the active pharmaceutical ingredients and excipients (per 200 kg of the total mixture).

1) UDCA+OCA—150 kg.

2) Microcrystalline cellulose—30 kg.

3) Lactose—18 kg.

4) Povidone—2 kg.

Step 2. Sifting. Sift the components through a sieve with a mesh size of at least 30 mesh.

Step 3. Dry mixing. Transfer the sifted components to a dry mixer and mix them for 1 hour to prepare a dry mixture.

Step 4: Compressing. Transfer the mixture produced in Step 3 to a compression machine, and compress into the solid dosage forms (tablets).

Step 5. Tablet coating. Transfer the tablets produced in Step 4 to a tablet film coating machine. Coat the tablets with the film by immersion in an ethanol solution of a film former (eudragit L).

### Example 2

A double-blind clinical study was conducted on the efficacy of the solid dosage forms comprising 2 APIs, UDCA and OCA, compared with the solid dosage forms comprising 1 API, UDCA, for patients with the values of the proposed group of the markers (as a percentage relative to the maximum value of the level of this marker in PBC):
IL-6—at least 10%;
Nf-kB—at least 5%;
MCP-1/CCL2—at least 5%;
Bcl-2—at least 5%;
TNF-α—at least 12%;
MDA—at least 0.5%.

IL-6 (Interleukin 6) is a pro-inflammatory interleukin synthesized by immune cells. It can stimulate the inflammatory process at the site of synthesis. An increase in the amount of IL-6 is observed in the early stages of the disease, when the autoimmune process is just beginning to develop. Its detection in liver biopsy specimens indicates an acute inflammatory process.

Nf-kB (nuclear factor kappa-light-chain-enhancer of activated B cells) is a protein complex that controls DNA transcription, cytokine synthesis and cell survival. NF-kB is found in almost all types of the animal cells and is involved in cellular responses to stimuli, such as stress, cytokines, free radicals, heavy metals, ultraviolet radiation, lipid peroxidation, and bacterial or viral antigens. NF-kB plays a key role in regulating the immune response. Misregulation of NF-kB is associated with the development of oncology, inflammatory and autoimmune diseases.

MCP-1/CCL2 (MCP-1, Monocyte chemoattractant protein-1) is one of the key chemokines that regulates the migration and infiltration of monocytes from peripheral blood into tissues. In PBC, it is released by Kupffer cells, and it stimulates the migration of monocytes from the peripheral blood to the liver tissue, which leads to further development of inflammation.

Bcl-2 is a mitochondrial apoptosis factor. Its elevated value indicates pathological cell death. This pathological phenomenon can be eliminated by neutralizing the primary cause of the cell death (in this case, this is the toxic effect of the bile acids and the autoimmune process).

TNF-α (tumor necrosis factor alpha) is a cytokine, i.e., a small protein used by the immune system for signaling. The primary role of this molecule is the regulation of the activity and metabolism of the immune cells. It can induce inflammation and apoptosis, which may be the cause of many autoimmune diseases. In PBC, an increase in the value of this cytokine is one of the early events; when immune cells are just starting to attack their own cells, its value rises. This happens much earlier than the rise in ALT/AST and bilirubin levels. That is, the detection of this marker in liver biopsy specimens indicates the beginning of the development of the inflammatory process.

MDA is a marker of inflammation, but from the lipid peroxidation point of view. Its increase indicates a high level of highly reactive oxygen in the tissues, which can be caused by both the inflammation and immune response.

Patients diagnosed with PBC were divided into 3 groups depending on the treatment regimen:
1 Treatment regimen: “UDCA 15 mg/kg” group. This group received solid dosage forms comprising 1 API, UDCA, in the amount of 15 mg of API per 1 kg of the patient body weight.
2 Treatment regimen: “UDCA 15 mg/kg+OCA 5 mg” group. This group received solid dosage forms according to Example 1, based on the patient body weight and a daily dose of OCA of 5 mg.
3 Treatment regimen: “UDCA 15 mg/kg+OCA 10 mg” group. This group received solid dosage forms according to Example 1, based on the patient body weight and a daily dose of OCA of 10 mg.

The initial characteristics of the groups are given in Table 2.

TABLE 2
Characteristics of the groups before the start of the study

| | | | |
|---|---|---|---|
| | Group | Group ″UDCA | Group ″UDCA |
| | ″UDCA 15 | 15 mg/kg + | 15 mg/kg + |
| | mg/kg″ | OCA 5 mg″ | OCA 10 mg″ |
| Parameter | (N = 68) | (N = 65) | (N = 63) |

Age

| | | | |
|---|---|---|---|
| Mean value (years) | 59.6 | 58.2 | 59.1 |
| Range (years) | 47-79 | 47-78 | 48-77 |

Sex

| | | | |
|---|---|---|---|
| Men (N) | 9 | 7 | 3 |
| Women (N) | 59 | 58 | 60 |

Laboratory markers (mean concentration level values)

| | | | |
|---|---|---|---|
| ALT (U/l) | 82.7 | 71.33 | 84.3 |
| AST (U/l) | 105.2 | 106.9 | 112.5 |
| GGT (U/l) | 207.5 | 212.3 | 208.8 |
| ALP (U/l) | 368 | 349 | 325 |
| Total bilirubin | 40.3 | 38.6 | 41.33 |
| (umol/l) | | | |
| Presence of AMA | 95 | 98 | 94 |
| antibodies (%) | | | |
| IgM (g/l) | 0.5 | 0.7 | 0.58 |
| TNF-α (ng/ml) | 0.33 | 0.35 | 0.33 |
| IL-6 (pg/ml) | 23.54 | 24.87 | 26.2 |
| Nf-KB (pg/ml) | 65.5 | 67.6 | 69.5 |
| MCP-1/CCL2 | 468.5 | 485.4 | 495.2 |
| (pg/ml) | | | |
| Bcl-2 (U/ml) | 0.72 | 0.69 | 0.71 |
| MDA (nmol/ml) | 23.08 | 22.58 | 24.01 |

Comparison of the effectiveness of the solid dosage forms comprising 2 APIs (UDCA and OCA), and solid dosage forms comprising one API (UDCA) was performed for 6 and 12 months.

After the treatment, venous blood samples were taken from the patients and the concentrations of biochemical and molecular markers were determined.

As markers of the treatment effectiveness, two groups of the markers were used: biochemical and molecular biological. The first group included: ALT, AST, GGT (Gamma-glutamyl Transferase), ALP (alkaline phosphatase), and total bilirubin. The second group included: TNF-α, IL-6, Nf-kB, MCP-1/CCL2, BCL-2, and MDA.

The study results after 6 months are shown in Tables 3-4.

TABLE 3
Concentration level values of the markers
in the blood plasma (6 months)

| | | | Group | | Group | |
|---|---|---|---|---|---|---|
| | Group | | “UDCA | | “UDCA | |
| | “UDCA | | 15 mg/kg + | | 15 mg/kg + | |
| | 15 mg/kg” | | OCA 5 mg” | | OCA 10 mg” | |
| | (N = 68) | | (N = 65) | | (N = 63) | |
| Parameter | Before | After | Before | After | Before | After |
| ALT (U/l) | 55.7 | 37.5 | 56.33 | 28.2 | 58.3 | 25.3 |
| AST (U/l) | 58.6 | 39.4 | 59.5 | 31.7 | 61.4 | 25.1 |
| GGT (U/l) | 207.5 | 135.2 | 212.3 | 74.2 | 208.8 | 60.2 |
| ALP (U/l) | 297.2 | 190 | 284.5 | 147.2 | 281.2 | 120.2 |
| Total | 57.6 | 20 | 59.8 | 12.8 | 54.3 | 12.1 |
| bilirubin | | | | | | |
| (μmol/l) | | | | | | |
| TNF-a | 0.33 | 0.29 | 0.35 | 0.123 | 0.33 | 0.1 |
| (ng/ml) | | | | | | |
| IL-6 (pg/ml) | 23.54 | 20.2 | 24.87 | 5.15 | 26.2 | 3.15 |
| Nf-kB (pg/ml) | 65.5 | 60.2 | 67.6 | 15.2 | 69.5 | 12.3 |
| MCP-1/CCL2 | 468.5 | 412.3 | 485.4 | 212.8 | 495.2 | 200.2 |
| (pg/ml) | | | | | | |
| Bcl-2 (U/ml) | 0.42 | 0.4 | 0.39 | 0.3 | 0.41 | 0.28 |
| MDA | 23.08 | 22.01 | 22.58 | 10.17 | 24.01 | 7.2 |
| (nmol/ml) | | | | | | |

TABLE 4
Changes of the marker values,
in % of the initial value (6 months)

| | | | Group | | Group | |
|---|---|---|---|---|---|---|
| | Group | | “UDCA | | “UDCA | |
| | “UDCA | | 15 mg/kg + | | 15 mg/kg + | |
| | 15 mg/kg” | | OCA 5 mg” | | OCA 10 mg” | |
| | (N = 68) | | (N = 65) | | (N = 63) | |
| Parameter | Before | After | Before | After | Before | After |
| ALT | 100 | 67.32 | 100 | 50.06 | 100 | 43.40 |
| AST | 100 | 67.24 | 100 | 53.28 | 100 | 40.88 |
| GGT | 100 | 65.16 | 100 | 34.95 | 100 | 28.83 |
| ALP | 100 | 63.93 | 100 | 51.74 | 100 | 42.75 |
| Total | 100 | 34.72 | 100 | 21.40 | 100 | 22.28 |
| bilirubin | | | | | | |
| TNF-α | 100 | 87.88 | 100 | 35.14 | 100 | 30.30 |
| IL-6 | 100 | 85.81 | 100 | 20.71 | 100 | 12.02 |
| Nf-kB | 100 | 91.91 | 100 | 22.49 | 100 | 17.70 |
| MCP-1/ | 100 | 88.00 | 100 | 43.84 | 100 | 40.43 |
| CCL2 | | | | | | |
| Bcl-2 | 100 | 95.24 | 100 | 76.92 | 100 | 68.29 |
| MDA | 100 | 95.36 | 100 | 45.04 | 100 | 29.99 |

Results of the study on the effectiveness of the treatment of the patients using the solid dosage forms comprising UDCA or UDCA+OCA after 12 months of the study are shown in the Tables 5-6.

TABLE 5
Concentration level values of the markers
in the blood plasma (12 months)

| | | | Group | | Group | |
|---|---|---|---|---|---|---|
| | Group | | “UDCA | | “UDCA | |
| | “UDCA | | 15 mg/kg + | | 15 mg/kg + | |
| | 15 mg/kg” | | OCA 5 mg” | | OCA 10 mg” | |
| | (N = 68) | | (N = 65) | | (N = 63) | |
| Parameter | Before | After | Before | After | Before | After |
| ALT (U/l) | 82.7 | 55.67 | 71.33 | 35.7 | 84.3 | 36.59 |
| AST (U/l) | 105.2 | 70.73 | 106.9 | 56.96 | 112.5 | 45.99 |
| GGT (U/l) | 207.5 | 135.2 | 212.3 | 74.2 | 208.8 | 60.2 |
| ALP (U/l) | 368.4 | 235.36 | 349 | 180.57 | 325 | 138.93 |
| Total | 40.3 | 26.03 | 38.6 | 14.74 | 41.33 | 9.2 |
| bilirubin | | | | | | |
| (μmol/l) | | | | | | |
| TNF-a | 0.33 | 0.29 | 0.35 | 0.123 | 0.33 | 0.1 |
| (ng/ml) | | | | | | |
| IL-6 (pg/ml) | 23.54 | 20.2 | 24.87 | 5.15 | 26.2 | 3.15 |
| Nf-kB (pg/ml) | 65.5 | 60.2 | 67.6 | 15.2 | 69.5 | 12.3 |
| MCP-1/ | 468.5 | 412.3 | 485.4 | 212.8 | 495.2 | 200.2 |
| CCL2 | | | | | | |
| (pg/ml) | | | | | | |
| Bcl-2 (U/ml) | 0.72 | 0.52 | 0.69 | 0.43 | 0.71 | 0.34 |
| MDA | 23.08 | 22.01 | 22.58 | 10.17 | 24.01 | 7.2 |
| (nmol/ml) | | | | | | |

TABLE 6
Changes of the marker values,
in % of the initial value (12 months)

| | | | Group | | Group | |
|---|---|---|---|---|---|---|
| | Group | | “UDCA | | “UDCA | |
| | “UDCA | | 15 mg/kg + | | 15 mg/kg + | |
| | 15 mg/kg” | | OCA 5 mg” | | OCA 10 mg” | |
| | (N = 68) | | (N = 65) | | (N = 63) | |
| Parameter | Before | After | Before | After | Before | After |
| ALT | 100 | 67.32 | 100 | 50.06 | 100 | 43.40 |
| AST | 100 | 67.24 | 100 | 53.28 | 100 | 40.88 |
| GGT | 100 | 65.16 | 100 | 34.95 | 100 | 28.83 |
| ALP | 100 | 63.93 | 100 | 51.74 | 100 | 42.75 |
| Total | 100 | 64.58 | 100 | 38.2 | 100 | 22.28 |
| bilirubin | | | | | | |
| TNF-α | 100 | 87.88 | 100 | 35.14 | 100 | 30.30 |
| IL-6 | 100 | 85.81 | 100 | 20.71 | 100 | 12.02 |
| Nf-kB | 100 | 91.91 | 100 | 22.49 | 100 | 17.70 |
| MCP-1/ | 100 | 88.00 | 100 | 43.84 | 100 | 40.43 |
| CCL2 | | | | | | |
| Bcl-2 | 100 | 72 | 100 | 62.32 | 100 | 47.89 |
| MDA | 100 | 95.36 | 100 | 45.04 | 100 | 29.99 |

The results obtained enable drawing the following conclusions:
1. The solid dosage forms comprising UDCA were more effective in reducing biochemical but not molecular biological markers of inflammation and apoptosis.
2. The effect of UDCA on the markers of inflammation, apoptosis and lipid peroxidation was insignificant.
3. The use of the solid dosage forms, comprising 2 APIs, UDCA and OCA, enables reducing not only biochemical markers, but also coping effectively with an acute inflammatory process and ceasing the cell death and development of fibrosis.

More detailed study results per months are given below in Tables 7-12.

TABLE 7
Changes in the concentration level values of the markers in the group
“UDCA 15 mg/kg” (0-12 months)

| | Months | | | | | |
|---|---|---|---|---|---|---|
| Parameter | 0 | 1 | 3 | 6 | 9 | 12 |
| ALT (U/l) | 82.7 | 80.4 | 74.81 | 68.2 | 62.47 | 55.67 |
| AST (U/l) | 105.2 | 100.7 | 87.3 | 81.4 | 74.5 | 70.73 |
| GGT (U/l) | 207.5 | 198 | 178.8 | 156.2 | 141.5 | 135.2 |
| ALP (U/l) | 368.4 | 352.5 | 290.6 | 269.45 | 247.5 | 235.36 |
| Total | 40.3 | 39.8 | 33.8 | 32.8 | 28.03 | 26.03 |
| bilirubin | | | | | | |
| (μmol/l) | | | | | | |
| TNF-α | 0.33 | 0.33 | 0.324 | 0.315 | 0.3 | 0.29 |
| (ng/ml) | | | | | | |
| IL-6 (pg/ml) | 23.54 | 23.35 | 22.95 | 22 | 21.48 | 20.2 |
| Nf-kB | 65.5 | 64.4 | 64.1 | 62.8 | 61.4 | 60.2 |
| (pg/ml) | | | | | | |
| MCP-1/ | 468.5 | 465.4 | 448 | 431.4 | 419.7 | 412.3 |
| CCL2 | | | | | | |
| (pg/ml) | | | | | | |
| Bcl-2 (U/ml) | 0.72 | 0.68 | 0.62 | 0.58 | 0.54 | 0.52 |
| MDA | 23.08 | 22.87 | 22.54 | 22.37 | 22.12 | 22.01 |
| (nmol/ml) | | | | | | |

TABLE 8
Changes in the concentration level values of the markers in the group
“UDCA 15 mg/kg” (0-12 months)

| | Months | | | | | |
|---|---|---|---|---|---|---|
| Parameter | 0 | 1 | 3 | 6 | 9 | 12 |
| ALT | 100.00 | 97.22 | 90.46 | 82.47 | 75.54 | 67.32 |
| AST | 100.00 | 95.72 | 82.98 | 77.38 | 70.82 | 67.23 |
| GGT | 100.00 | 95.42 | 86.17 | 75.28 | 68.19 | 65.16 |
| ALP | 100.00 | 97.20 | 88.10 | 74.45 | 69.18 | 63.93 |
| Total | 100.00 | 96.18 | 87.50 | 77.60 | 70.49 | 64.58 |
| bilirubin | | | | | | |
| TNF-α | 100.00 | 100.00 | 98.18 | 95.45 | 90.91 | 87.88 |
| IL-6 | 100.00 | 99.19 | 97.49 | 93.46 | 91.25 | 85.81 |
| Nf-kB | 100.00 | 98.32 | 97.86 | 95.88 | 93.74 | 91.91 |
| MCP-1/ | 100.00 | 99.34 | 95.62 | 92.08 | 89.58 | 88.00 |
| CCL2 | | | | | | |
| Bcl-2 | 100.00 | 94.44 | 86.11 | 80.56 | 75.00 | 72.22 |
| MDA | 100.00 | 99.09 | 97.66 | 96.92 | 95.84 | 95.36 |

Analysis of the obtained data shows that in patients of the “UDCA 15 mg/kg” group, the most effective was reduction of the markers, such as ALT, AST, GGT, ALP, total bilirubin and BCL-2, which indicates that such a treatment regimen obtained by this group is somewhat effective, but it had little effect on inflammation processes.

TABLE 9
Changes in the concentration level values of the markers in the group
“UDCA 15 mg/kg + OCA 5 mg” (0-12 months)

| | Months | | | | | |
|---|---|---|---|---|---|---|
| Parameter | 0 | 1 | 3 | 6 | 9 | 12 |
| ALT (U/l) | 71.33 | 69.7 | 61.7 | 52.75 | 41.79 | 35.7 |
| AST (U/l) | 106.9 | 101.4 | 87 | 79.45 | 71.7 | 56.96 |
| GGT (U/l) | 212.3 | 207.4 | 184.4 | 134.6 | 87.5 | 74.2 |
| ALP (U/l) | 349 | 332.3 | 285.6 | 245.8 | 210.5 | 180.57 |
| Total | 38.6 | 37.6 | 31.05 | 24.8 | 16.87 | 14.74 |
| bilirubin | | | | | | |
| (μmol/l) | | | | | | |
| TNF-α | 0.35 | 0.27 | 0.24 | 0.178 | 0.14 | 0.123 |
| (ng/ml) | | | | | | |
| IL-6 (pg/ml) | 24.87 | 18.6 | 16.7 | 12.6 | 8.9 | 5.15 |
| Nf-kB | 67.6 | 59.7 | 41.5 | 29.7 | 19.4 | 15.2 |
| (pg/ml) | | | | | | |
| MCP-1/ | 485.4 | 403.6 | 301.5 | 270 | 235.4 | 212.8 |
| CCL2 | | | | | | |
| (pg/ml) | | | | | | |
| Bcl-2 (U/ml) | 0.69 | 0.67 | 0.62 | 0.51 | 0.45 | 0.43 |
| MDA | 22.58 | 19.68 | 17.8 | 13.8 | 12.6 | 10.17 |
| (nmol/ml) | | | | | | |

TABLE 10
Changes in the concentration level values of the markers in the group
“UDCA 15 mg/kg + OCA 5 mg” (0-12 months)

| | Months | | | | | |
|---|---|---|---|---|---|---|
| Parameter | 0 | 1 | 3 | 6 | 9 | 12 |
| ALT | 100.00 | 97.71 | 86.50 | 73.95 | 58.59 | 50.05 |
| AST | 100.00 | 94.86 | 81.38 | 74.32 | 67.07 | 53.28 |
| GGT | 100.00 | 97.69 | 86.86 | 63.40 | 41.22 | 34.95 |
| ALP | 100.00 | 95.60 | 84.91 | 70.29 | 57.32 | 51.74 |
| Total bilirubin | 100.00 | 98.16 | 76.76 | 59.87 | 49.16 | 38.19 |
| TNF-α | 100.00 | 77.14 | 68.57 | 50.86 | 40.00 | 35.14 |
| IL-6 | 100.00 | 74.79 | 67.15 | 50.66 | 35.79 | 20.71 |
| Nf-kB | 100.00 | 88.31 | 61.39 | 43.93 | 28.70 | 22.49 |
| MCP-1/CCL2 | 100.00 | 83.15 | 62.11 | 55.62 | 48.50 | 43.84 |
| Bcl-2 | 100.00 | 97.10 | 89.86 | 73.91 | 65.22 | 62.32 |
| MDA | 100.00 | 87.16 | 78.83 | 61.12 | 55.80 | 45.04 |

In the “UDCA 15 mg/kg+OCA 5 mg” group, a more significant decrease in biochemical markers characterizing the general functional condition of the liver was observed, there was also a significant decrease in the markers of inflammation, chemoattraction of monocytes from peripheral blood, apoptosis and lipid peroxidation. Thus, it can be concluded that the treatment regimen received by the “UDCA 15 mg/kg+OCA 5 mg” group was more efficient compared with the treatment regimen received by the “UDCA 15 mg/kg” group.

TABLE 11
Changes in the concentration level values of the markers in
“UDCA 15 mg/kg + OCA 10 mg” group (0-12 months)

| | Months | | | | | |
|---|---|---|---|---|---|---|
| | 0 | 1 | 3 | 6 | 9 | 12 |
| ALT (U/l) | 84.3 | 82.4 | 71.9 | 59.1 | 41.8 | 36.59 |
| AST (U/l) | 112.5 | 109.6 | 85.6 | 69.96 | 51.8 | 45.99 |
| GGT (U/l) | 208.8 | 198.6 | 135 | 106 | 89.5 | 60.2 |
| ALP (U/l) | 325 | 319.5 | 289.57 | 210.5 | 178.57 | 138.93 |
| Total bilirubin | 41.33 | 39.25 | 31.05 | 24.6 | 16.8 | 9.2 |
| (μmol/l) | | | | | | |
| TNF-α | 0.33 | 0.28 | 0.25 | 0.18 | 0.13 | 0.1 |
| (ng/ml) | | | | | | |
| IL-6 (pg/ml) | 26.2 | 21.3 | 16.9 | 13.69 | 7.45 | 3.15 |
| Nf-kB (pg/ml) | 69.5 | 57.61 | 35.6 | 22 | 14.6 | 12.3 |
| MCP-1/CCL2 | 495.2 | 401.7 | 301.8 | 270.2 | 230.8 | 200.2 |
| (pg/ml) | | | | | | |
| Bcl-2 (U/ml) | 0.71 | 0.68 | 0.64 | 0.52 | 0.46 | 0.34 |
| MDA | 24.01 | 21.5 | 18.6 | 13.8 | 7.92 | 7.2 |
| (nmol/ml) | | | | | | |

TABLE 12
Changes in the marker concentration values in “UDCA 15 mg/kg +
OCA 10 mg” group, in % of the initial value (0-12 months)

| | Months | | | | | |
|---|---|---|---|---|---|---|
| | 0 | 1 | 3 | 6 | 9 | 12 |
| ALT | 100.00 | 97.75 | 85.29 | 70.11 | 49.58 | 43.40 |
| AST | 100.00 | 97.42 | 76.09 | 62.19 | 46.04 | 40.88 |
| GGT | 100.00 | 95.11 | 64.66 | 50.77 | 42.86 | 28.83 |
| ALP | 100.00 | 98.67 | 74.32 | 62.19 | 47.21 | 42.75 |
| Total bilirubin | 100.00 | 96.50 | 65.56 | 36.10 | 29.10 | 22.28 |
| TNF-α | 100.00 | 84.85 | 75.76 | 54.55 | 39.39 | 30.30 |
| IL-6 | 100.00 | 81.30 | 64.50 | 52.25 | 28.44 | 12.02 |
| Nf-kB | 100.00 | 82.89 | 51.22 | 31.65 | 21.01 | 17.70 |
| MCP-1/CCL2 | 100.00 | 81.12 | 60.95 | 54.56 | 46.61 | 40.43 |
| Bcl-2 | 100.00 | 95.77 | 90.14 | 73.24 | 64.79 | 47.89 |
| MDA | 100.00 | 89.55 | 77.47 | 57.48 | 32.99 | 29.99 |

In this group, there was a significant decrease in the biochemical markers characterizing the general functional condition of the liver, as well as a significant decrease in the markers of inflammation, chemoattraction of monocytes from peripheral blood, apoptosis and lipid peroxidation. It can be concluded that the treatment regimen received by the “UDCA 15 mg/kg+OCA 10 mg” group was more efficient compared with the treatment regimens received by the “UDCA 15 mg/kg” and “UDCA 15 mg/kg+OCA 5 mg” groups.

When all three treatment regimens are compared, it is observed that:
1) The solid dosage form comprising UDCA has a specific effect on the overall functional condition of the liver, but almost no effect on the inflammatory and immunological component of the disease.
2) The solid dosage form comprising UDCA and OCA is more efficient in the treatment of PBC, because it affects not only the general functional condition of the liver, but also the molecular mechanisms of the development of this disease, that is, the treatment is not only symptomatic.

Based on the conducted studies, the authors consider it necessary to use the solid dosage form for the treatment of PBC, the active pharmaceutical ingredients of which are 2APIs: UDCA, that stimulates the excretion of bile acids; and OCA, that stimulates the nuclear FXR receptor, which leads to inhibition of bile acid synthesis, anti-inflammatory, antifibrotic and antiapoptotic effect of the solid dosage form.

### Example 3

To extend the investigation, further studies examined possible challenges in incorporating two APIs in a single immediate-release dosage form, particularly unwanted API interactions, in continuity with the initial assessment.

A study evaluated the pharmacokinetics of OCA after oral administration, both in solid dosage forms comprising only OCA and in solid dosage forms comprising OCA and UDCA.

The study included solid dosage forms with the characteristics described as follows:
Sample 1 is an immediate release solid dosage form comprising 5 mg OCA.
Sample 2 is an immediate release solid dosage form comprising 10 mg OCA.
Sample 3 is an immediate release solid dosage form comprising 5 mg OCA and 500 mg UDCA.
Sample 4 is an immediate release solid dosage form comprising 10 mg OCA and 500 mg UDCA.
Sample 5 is a modified release solid dosage form comprising 5 mg OCA, 500 mg UDCA, and enteric coating.
Sample 6 is a modified release solid dosage form comprising 10 mg OCA, 500 mg UDCA, and enteric coating.

The results of the pharmacokinetic studies are presented in Table 13.

TABLE 13
Pharmacokinetic parameters of the solid
dosage forms with OCA and UDCA

| | Sample No. | | | | | |
|---|---|---|---|---|---|---|
| Parameter | 1 | 2 | 3 | 4 | 5 | 6 |
| Cmax (ng/ml) | 16.7 | 26.4 | 7.1 | 14.8 | 16.2 | 26.8 |
| AUCt (ng*h/ml) | 34.8 | 66.1 | 19.4 | 35.8 | 33.7 | 65.8 |
| Tmax (min) | 40 | 90 | 95 | 140 | 45 | 87 |
| Bioavailability | 89 | 91 | 56 | 61 | 87 | 88 |
| (%) | | | | | | |

It was unexpectedly found that when OCA is taken simultaneously with UDCA, UDCA suppresses the absorption of OCA, reducing bioavailability from 89% to 56% for 5 mg OCA, and from 91% to 61% for 10 mg OCA, respectively.

The development of a new all-purpose solid dosage form for the treatment of PBC, comprising both UDCA and OCA and characterized by a complex mechanism of action, allows effective treatment of all stages of PBC. In particular, the solid dosage form enables simultaneous inhibition of bile acid transport and synthesis, resulting in a pronounced hepatoprotective effect.

Taking into account the obtained experimental data on potential absorption conflicts between the two APIs, it was concluded that achieving the desired therapeutic effectiveness of the solid dosage form for the treatment of PBC requires a more sophisticated approach. As a result of the conducted studies, a modified release solid dosage form comprising UDCA and OCA was developed, providing improved pharmacokinetic performance and therapeutic effect.

### Example 4

According to one aspect, the authors of the present disclosure suggest the manufacture of the modified release solid dosage form comprising OCA and UDCA in the form of a multilayer tablet, wherein the release of OCA occurs first, and then the release of UDCA occurs.

According to the present disclosure, the modified release solid dosage form comprises a core comprising ursodeoxycholic acid; and at least one layer comprising obeticholic acid. Preferably, the modified release solid dosage form comprises the core and three layers, wherein the core comprises ursodeoxycholic acid and at least one of the layers comprises obeticholic acid.

According to one of the embodiments of the present disclosure the modified release solid dosage form comprises:
(a) The outer layer comprising the enteric coating (achieved by implementing specific polymers).
(b) The second layer comprising obeticholic acid. According to one of the embodiments, after the dissolution of the outer layer, the release and absorption of OCA occurs in the intestinal lumen.
(c) The third layer comprising the STOP layer. The STOP layer is a special layer, the swelling and dissolution of which takes about 1-2 hours, which allows the OCA to be completely absorbed.
(d) The core comprising ursodeoxycholic acid. After the dissolution of all the above layers, UDCA is released and absorbed. According to one of the embodiments, the core can be designed with extended release of UDCA (with a release time of up to 6 hours).

According to another embodiment of the present disclosure, the modified release solid dosage form comprises:
(a) The outer layer comprising the gastric acid soluble layer.
(b) The second layer comprising obeticholic acid. After the outer layer dissolves, the OCA is released and absorbed.
(c) The third layer comprising the enteric coating. The layer is made resistant to the action of gastric acid (due to special polymers).
(d) The core comprising ursodeoxycholic acid. After the dissolution of all the above layers, UDCA is released and absorbed. According to one of the embodiments, the core can be designed with extended release of UDCA (with release time up to 6 hours).

A further advantage of incorporating OCA and UDCA into a single solid dosage form, especially a tablet of defined structure, lies in achieving enhanced treatment compliance. The need for the patient to take two separate drugs, given the complexity of the treatment regimen (the number of solid dosage forms and the number of doses) and the specificity of PBC symptoms with the manifestation of fatigue and lack of concentration, will lead to errors in administering the drugs: both in the number of the tablets and in administering them over time. Moreover, the authors of this disclosure were the first to study the problem of competition between OCA and UDCA for the transporters, which is an important factor that has not been considered previously in this context. To achieve the best efficacy of the PBC treatment, it is necessary to achieve an optimal regimen for the administration of two APIs, which will not suppress the absorption of OCA due to the untimely administration of the second drug with UDCA.

Thus, using the multilayer tablet, separate release of the acids is achieved, while their competition is reduced and the bioavailability of the APIs is improved. Some possible embodiments of the claimed solid dosage forms in the form of multilayered tablets are presented in Table 14.

TABLE 14
Embodiments of modified release solid dosage forms
in the multilayer tablet form

| Embodiment No. | Description | Structure |
|---|---|---|
| Embodiment 1 | Coated multilayer modified | Outer layer-enteric coating |
| | release tablet | Second layer-OCA-1 mg |
| | | Third layer-STOP layer |
| | | Core-UDCA-100 mg |
| Embodiment 2 | Coated multilayer modified | Outer layer-gastric acid |
| | release tablet with extended | soluble layer-film coating |
| | release of UDCA | Second layer-OCA-2.5 mg |
| | | Third layer-STOP layer |
| | | Core-UDCA-100 mg |
| Embodiment 3 | Coated multilayer modified | Outer layer-enteric coating |
| | release tablet | Second layer-OCA-5 mg |
| | | Third layer-STOP layer |
| | | Core-UDCA-100 mg |
| Embodiment 4 | Coated multilayer modified | Outer layer-gastric acid |
| | release tablet with extended | soluble layer-film coating |
| | release of UDCA | Second layer-OCA-10 mg |
| | | Third layer-STOP layer |
| | | Core-UDCA-100 mg |
| Embodiment 5 | Coated multilayer modified | Outer layer-enteric coating |
| | release tablet | Second layer-OCA-20 mg |
| | | Third layer-STOP layer |
| | | Core-UDCA-100 mg |
| Embodiment 6 | Coated multilayer modified | Outer layer-gastric acid |
| | release tablet with extended | soluble layer-film coating |
| | release of UDCA | Second layer-OCA-50 mg |
| | | Third layer-STOP layer |
| | | Core-UDCA-100 mg |
| Embodiment 7 | Coated multilayer modified | Outer layer-enteric coating |
| | release tablet | Second layer-OCA-1 mg |
| | | Third layer-STOP layer |
| | | Core-UDCA-250 mg |
| Embodiment 8 | Coated multilayer modified | Outer layer-gastric acid |
| | release tablet with extended | soluble layer-film coating |
| | release of UDCA | Second layer-OCA-2.5 mg |
| | | Third layer-STOP layer |
| | | Core-UDCA-250 mg |
| Embodiment 9 | Coated multilayer modified | Outer layer-enteric coating |
| | release tablet | Second layer-OCA-5 mg |
| | | Third layer-STOP layer |
| | | Core-UDCA-250 mg |
| Embodiment 10 | Coated multilayer modified | Outer layer-gastric acid |
| | release tablet with extended | soluble layer-film coating |
| | release of UDCA | Second layer-OCA-10 mg |
| | | Third layer-STOP layer |
| | | Core-UDCA-250 mg |
| Embodiment 11 | Coated multilayer modified | Outer layer-enteric coating |
| | release tablet | Second layer-OCA-20 mg |
| | | Third layer-STOP layer |
| | | Core-UDCA-250 mg |
| Embodiment 12 | Coated multilayer modified | Outer layer-gastric acid |
| | release tablet with extended | soluble layer-film coating |
| | release of UDCA | Second layer-OCA-50 mg |
| | | Third layer-STOP layer |
| | | Core-UDCA-250 mg |
| Embodiment 13 | Coated multilayer modified | Outer layer-enteric coating |
| | release tablet | Second layer-OCA-1 mg |
| | | Third layer-STOP layer |
| | | Core-UDCA-500 mg |
| Embodiment 14 | Coated multilayer modified | Outer layer-gastric acid |
| | release tablet with extended | soluble layer-film coating |
| | release of UDCA | Second layer-OCA-2.5 mg |
| | | Third layer-STOP layer |
| | | Core-UDCA-500 mg |
| Embodiment 15 | Coated multilayer modified | Outer layer-enteric coating |
| | release tablet | Second layer-OCA-5 mg |
| | | Third layer-STOP layer |
| | | Core-UDCA-500 mg |
| Embodiment 16 | Coated multilayer modified | Outer layer-gastric acid |
| | release tablet with extended | soluble layer-film coating |
| | release of UDCA | Second layer-OCA-10 mg |
| | | Third layer-STOP layer |
| | | Core-UDCA-500 mg |
| Embodiment 17 | Coated multilayer modified | Outer layer-enteric coating |
| | release tablet | Second layer-OCA-20 mg |
| | | Third layer-STOP layer |
| | | Core-UDCA-500 mg |
| Embodiment 18 | Coated multilayer modified | Outer layer-gastric acid |
| | release tablet with extended | soluble layer-film coating |
| | release of UDCA | Second layer-OCA-5 mg |
| | | Third layer-STOP layer |
| | | Core-UDCA-500 mg |
| Embodiment 19 | Coated multilayer modified | Outer layer-enteric coating |
| | release tablet | Second layer-OCA-1 mg |
| | | Third layer-STOP layer |
| | | Core-UDCA-1000 mg |
| Embodiment 20 | Coated multilayer modified | Outer layer-gastric acid |
| | release tablet with extended | soluble layer-film coating |
| | release of UDCA | Second layer-OCA-2.5 mg |
| | | Third layer-STOP layer |
| | | Core-UDCA-1000 mg |
| Embodiment 21 | Coated multilayer modified | Outer layer-enteric coating |
| | release tablet | Second layer-OCA-5 mg |
| | | Third layer-STOP layer |
| | | Core-UDCA-1000 mg |
| Embodiment 22 | Coated multilayer modified | Outer layer-gastric acid |
| | release tablet with extended | soluble layer-film coating |
| | release of UDCA | Second layer-OCA-10 mg |
| | | Third layer-STOP layer |
| | | Core-UDCA-1000 mg |
| Embodiment 23 | Coated multilayer modified | Outer layer-enteric coating |
| | release tablet | Second layer-OCA-20 mg |
| | | Third layer-STOP layer |
| | | Core-UDCA-1000 mg |
| Embodiment 24 | Coated multilayer modified | Outer layer-gastric acid |
| | release tablet with extended | soluble layer-film coating |
| | release of UDCA | Second layer-OCA-50 mg |
| | | Third layer-STOP layer |
| | | Core-UDCA-1000 mg |
| Embodiment 25 | Coated multilayer modified | Outer layer-gastric acid |
| | release tablet | soluble layer-film coating |
| | | Second layer-OCA-1 mg |
| | | Third layer-enteric coating |
| | | Core-UDCA-100 mg |
| Embodiment 26 | Coated multilayer modified | Outer layer-gastric acid |
| | release tablet with extended | soluble layer-film coating |
| | release of UDCA | Second layer-OCA-2.5 mg |
| | | Third layer-enteric coating |
| | | Core-UDCA-100 mg |
| Embodiment 27 | Coated multilayer modified | Outer layer-gastric acid |
| | release tablet | soluble layer-film coating |
| | | Second layer-OCA-5 mg |
| | | Third layer-enteric coating |
| | | Core-UDCA-100 mg |
| Embodiment 28 | Coated multilayer modified | Outer layer-gastric acid |
| | release tablet with extended | soluble layer-film coating |
| | release of UDCA | Second layer-OCA-10 mg |
| | | Third layer-enteric coating |
| | | Core-UDCA-100 mg |
| Embodiment 29 | Coated multilayer modified | Outer layer-gastric acid |
| | release tablet | soluble layer-film coating |
| | | Second layer-OCA-20 mg |
| | | Third layer-enteric coating |
| | | Core-UDCA-100 mg |
| Embodiment 30 | Coated multilayer modified | Outer layer-gastric acid |
| | release tablet with extended | soluble layer-film coating |
| | release of UDCA | Second layer-OCA-50 mg |
| | | Third layer-enteric coating |
| | | Core-UDCA-100 mg |
| Embodiment 31 | Coated multilayer modified | Outer layer-gastric acid |
| | release tablet | soluble layer-film coating |
| | | Second layer-OCA-1 mg |
| | | Third layer-enteric coating |
| | | Core-UDCA-250 mg |
| Embodiment 32 | Coated multilayer modified | Outer layer-gastric acid |
| | release tablet with extended | soluble layer-film coating |
| | release of UDCA | Second layer-OCA-2.5 mg |
| | | Third layer-enteric coating |
| | | Core-UDCA-250 mg |
| Embodiment 33 | Coated multilayer modified | Outer layer-gastric acid |
| | release tablet | soluble layer-film coating |
| | | Second layer-OCA-5 mg |
| | | Third layer-enteric coating |
| | | Core-UDCA-250 mg |
| Embodiment 34 | Coated multilayer modified | Outer layer-gastric acid |
| | release tablet with extended | soluble layer-film coating |
| | release of UDCA | Second layer-OCA-10 mg |
| | | Third layer-enteric coating |
| | | Core-UDCA-250 mg |
| Embodiment 35 | Coated multilayer modified | Outer layer-gastric acid |
| | release tablet | soluble layer-film coating |
| | | Second layer-OCA-20 mg |
| | | Third layer-enteric coating |
| | | Core-UDCA-250 mg |
| Embodiment 36 | Coated multilayer modified | Outer layer-gastric acid |
| | release tablet with extended | soluble layer-film coating |
| | release of UDCA | Second layer-OCA-50 mg |
| | | Third layer-enteric coating |
| | | Core-UDCA-250 mg |
| Embodiment 37 | Coated multilayer modified | Outer layer-gastric acid |
| | release tablet | soluble layer-film coating |
| | | Second layer-OCA-1 mg |
| | | Third layer-enteric coating |
| | | Core-UDCA-500 mg |
| Embodiment 38 | Coated multilayer modified | Outer layer-gastric acid |
| | release tablet with extended | soluble layer-film coating |
| | release of UDCA | Second layer-OCA-2.5 mg |
| | | Third layer-enteric coating |
| | | Core-UDCA-500 mg |
| Embodiment 39 | Coated multilayer modified | Outer layer-gastric acid |
| | release tablet | soluble layer-film coating |
| | | Second layer-OCA-5 mg |
| | | Third layer-enteric coating |
| | | Core-UDCA-500 mg |
| Embodiment 40 | Coated multilayer modified | Outer layer-gastric acid |
| | release tablet with extended | soluble layer-film coating |
| | release of UDCA | Second layer-OCA-10 mg |
| | | Third layer-enteric coating |
| | | Core-UDCA-500 mg |
| Embodiment 41 | Coated multilayer modified | Outer layer-gastric acid |
| | release tablet | soluble layer-film coating |
| | | Second layer-OCA-20 mg |
| | | Third layer-enteric coating |
| | | Core-UDCA-500 mg |
| Embodiment 42 | Coated multilayer modified | Outer layer-gastric acid |
| | release tablet with extended | soluble layer-film coating |
| | release of UDCA | Second layer-OCA-5 mg |
| | | Third layer-enteric coating |
| | | Core-UDCA-500 mg |
| Embodiment 43 | Coated multilayer modified | Outer layer-gastric acid |
| | release tablet | soluble layer-film coating |
| | | Second layer-OCA-1 mg |
| | | Third layer-enteric coating |
| | | Core-UDCA-1000 mg |
| Embodiment 44 | Coated multilayer modified | Outer layer-gastric acid |
| | release tablet with extended | soluble layer-film coating |
| | release of UDCA | Second layer-OCA-2.5 mg |
| | | Third layer-enteric coating |
| | | Core-UDCA-1000 mg |
| Embodiment 45 | Coated multilayer modified | Outer layer-gastric acid |
| | release tablet | soluble layer-film coating |
| | | Second layer-OCA-5 mg |
| | | Third layer-enteric coating |
| | | Core-UDCA-1000 mg |
| Embodiment 46 | Coated multilayer modified | Outer layer-gastric acid |
| | release tablet with extended | soluble layer-film coating |
| | release of UDCA | Second layer-OCA-10 mg |
| | | Third layer-enteric coating |
| | | Core-UDCA-1000 mg |
| Embodiment 47 | Coated multilayer modified | Outer layer-gastric acid |
| | release tablet | soluble layer-film coating |
| | | Second layer-OCA-20 mg |
| | | Third layer-enteric coating |
| | | Core-UDCA-1000 mg |
| Embodiment 48 | Coated multilayer modified | Outer layer-gastric acid |
| | release tablet with extended | soluble layer-film coating |
| | release of UDCA | Second layer-OCA-50 mg |
| | | Third layer-enteric coating |
| | | Core-UDCA-1000 mg |

As shown in the following examples, according to some embodiments of the present disclosure, the claimed solid dosage form may have the following composition per tablet (UDCA+OCA and excipients):

Emdodiment 50

| | Quantity per |
|---|---|
| Ingredient | Tablet (mg) |
| API content, mg (UDCA + OCA) | 500 + 25 |

Core comprising UDCA

| | |
|---|---|
| Ursodeoxycholic acid | 500 |
| Disodium Phosphate | 50 |
| Polyvinylpyrrolidone | 24 |
| Microcrystalline Cellulose | 150 |
| Magnesium Stearate | 10 |
| Talc | 6 |
| Hydroxypropylmethylcellulose | 14 |
| PEG 6000 | 10 |
| Titanium Dioxide | 2 |
| Cellulose Acetate Phthalate | 24 |
| Acetylated Monoglycerides | 4 |

STOP Layer

| | |
|---|---|
| Microcrystalline Cellulose | 12 |
| Corn starch | 6 |
| Crospovidone | 4 |
| Polyvinylpyrrolidone | 4 |

Second layer Comprising OCA

| | |
|---|---|
| Obeticholic acid | 25 |
| Microcrystalline Cellulose | 29 |
| Sodium Starch Glycollate | 8 |
| Colloidal Silicon Dioxide | 2 |
| Magnesium Stearate | 4 |

Gastric acid soluble coating

| | |
|---|---|
| Opadry (ready-to-use film coating composition) | 12 |

Emdodiment 51

| | Quantity per |
|---|---|
| Ingredient | Tablet (mg) |
| API content, mg (UDCA + OCA) | 100 + 5 |

Core comprising UDCA

| | |
|---|---|
| Ursodeoxycholic acid | 100 |
| L Arginine | 10 |
| Polyvinylpyrrolidone | 10 |
| Microcrystalline Cellulose | 40 |
| Magnesium Stearate | 2 |
| Talc | 2 |
| Hydroxypropylmethylcellulose | 6 |
| PEG 6000 | 2 |
| Titanium Dioxide | 1 |
| Eudragit L100 | 6 |
| Acetylated Monoglycerides | 1 |

STOP Layer

| | |
|---|---|
| Microcrystalline Cellulose | 17 |
| Corn starch | 6 |
| Crospovidone | 2 |
| Polyvinylpyrrolidone | 3 |

Second layer Comprising OCA

| | |
|---|---|
| Obeticholic acid | 5 |
| Microcrystalline Cellulose | 20 |
| Sodium Starch Glycollate | 2 |
| Colloidal Silicon Dioxide | 1 |
| Magnesium Stearate | 2 |

Gastric acid soluble coating

| | |
|---|---|
| Opadry (ready-to-use film coating composition) | 12 |

Emdodiment 52

| | Quantity per |
|---|---|
| Ingredient | Tablet (mg) |
| API content, mg (UDCA + OCA) | 200 + 10 |

Core comprising UDCA

| | |
|---|---|
| Ursodeoxycholic acid | 200 |
| Sodium Maleate | 20 |
| Polyvinylpyrrolidone | 20 |
| Microcrystalline Cellulose | 80 |
| Magnesium Stearate | 4 |
| Talc | 4 |
| Ethyl Cellulose | 12 |
| PEG 6000 | 4 |
| Titanium Dioxide | 2 |
| Eudragit L100 | 12 |
| Acetylated Monoglycerides | 2 |

STOP Layer

| | |
|---|---|
| Microcrystalline Cellulose | 30 |
| Corn starch | 10 |
| Crospovidone | 4 |
| Polyvinylpyrrolidone | 6 |

Second layer Comprising OCA

| | |
|---|---|
| Obeticholic acid | 20 |
| Microcrystalline Cellulose | 36 |
| Sodium Starch Glycollate | 4 |
| Colloidal Silicon Dioxide | 2 |
| Magnesium Stearate | 4 |

Gastric acid soluble coating

| | |
|---|---|
| Opadry (ready-to-use film coating composition) | 24 |

Emdodiment 53

| | Quantity per |
|---|---|
| Ingredient | Tablet (mg) |
| API content, mg (UDCA + OCA) | 100 + 5 |

Core comprising UDCA

| | |
|---|---|
| Ursodeoxycholic acid | 100 |
| Disodium Phosphate | 10 |
| Polyvinylpyrrolidone | 10 |
| Microcrystalline Cellulose | 40 |
| Magnesium Stearate | 2 |
| Talc | 2 |
| Hydroxy Ethyl Cellulose | 6 |
| PEG 6000 | 2 |
| Titanium Dioxide | 1 |
| Eudragit L55D (EQ to dried solid | 6 |
| content) | |
| Acetylated Monoglycerides | 1 |

STOP Layer

| | |
|---|---|
| Microcrystalline Cellulose | 17 |
| Corn starch | 6 |
| Crospovidone | 2 |
| Polyvinylpyrrolidone | 3 |

Second layer Comprising OCA

| | |
|---|---|
| Obeticholic acid | 5 |
| Microcrystalline Cellulose | 20 |
| Sodium Starch Glycollate | 2 |
| Colloidal Silicon Dioxide | 1 |
| Magnesium Stearate | 2 |

Gastric acid soluble coating

| | |
|---|---|
| Opadry (ready-to-use film coating composition) | 12 |

Emdodiment 54

| | Quantity per |
|---|---|
| Ingredient | Tablet (mg) |
| API content, mg (UDCA + OCA) | 250 + 12.5 |

Core comprising UDCA

| | |
|---|---|
| Ursodeoxycholic acid | 250 |
| Sodium Bicarbobate | 25 |
| Polyvinylpyrrolidone | 12 |
| Microcrystalline Cellulose | 75 |
| Magnesium Stearate | 5 |
| Talc | 3 |
| Hydroxypropylmethylcellulose | 7 |
| PEG 6000 | 5 |
| Titanium Dioxide | 1 |
| Hydroxypropylmethylcellulose Acetyl | 12 |
| Succinate | |
| Acetylated Monoglycerides | 2 |

STOP Layer

| | |
|---|---|
| Microcrystalline Cellulose | 3 |
| Corn starch | 1 |
| Crospovidone | 2 |
| Polyvinylpyrrolidone | 2 |

Second layer Comprising OCA

| | |
|---|---|
| Obeticholic acid | 12.5 |
| Microcrystalline Cellulose | 20 |
| Sodium Starch Glycollate | 4 |
| Colloidal Silicon Dioxide | 0.5 |
| Magnesium Stearate | 2 |

Gastric acid soluble coating

| | |
|---|---|
| Opadry (ready-to-use film coating composition) | 6 |

Emdodiment 55

| | Quantity per |
|---|---|
| Ingredient | Tablet (mg) |
| API content, mg (UDCA + OCA) | 1000 + 50 |

Core comprising UDCA

| | |
|---|---|
| Ursodeoxycholic acid | 1000 |
| Sodium Bicarbobate | 100 |
| Polyvinylpyrrolidone | 30 |
| Microcrystalline Cellulose | 300 |
| Magnesium Stearate | 20 |
| Talc | 13 |
| Hydroxypropylmethylcellulose | 28 |
| PEG 6000 | 20 |
| Titanium Dioxide | 5 |
| Hydroxypropylmethylcellulose Phthalate | 50 |
| Acetylated Monoglycerides | 6 |

STOP Layer

| | |
|---|---|
| Microcrystalline Cellulose | 30 |
| Corn starch | 15 |
| Crospovidone | 8 |
| Polyvinylpyrrolidone | 8 |

Second layer Comprising OCA

| | |
|---|---|
| Obeticholic acid | 50 |
| Microcrystalline Cellulose | 80 |
| Sodium Starch Glycollate | 8 |
| Colloidal Silicon Dioxide | 3 |
| Magnesium Stearate | 2 |

Gastric acid soluble coating

| | |
|---|---|
| Opadry (ready-to-use film coating composition) | 24 |

Examples illustrating the preparation of the multilayer tablets according to the invention are provided below. The amounts of the components in each multilayer tablet are shown in the corresponding tables of each example. To prepare one hundred tablets, the mass of each component was multiplied by one hundred.

### Example 5

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 100 + 1 |

Core comprising UDCA

| | |
|---|---|
| Ursodeoxycholic acid | 100 |
| L Arginine | 10 |
| Polyvinylpyrrolidone | 10 |
| Microcrystalline Cellulose | 40 |
| Magnesium Stearate | 2 |
| Talc | 2 |
| Hydroxypropylmethylcellulose | 6 |
| PEG 6000 | 2 |
| Titanium Dioxide | 1 |
| Eudragit L100 | 6 |
| Acetylated Monoglycerides | 1 |

STOP Layer

| | |
|---|---|
| Microcrystalline Cellulose | 17 |
| Corn starch | 6 |
| Crospovidone | 2 |
| Polyvinylpyrrolidone | 3 |

Second layer Comprising OCA

| | |
|---|---|
| Obeticholic acid | 1 |
| Microcrystalline Cellulose | 20 |
| Sodium Starch Glycollate | 2 |
| Colloidal Silicon Dioxide | 1 |
| Magnesium Stearate | 2 |

Gastric acid soluble layer

| | |
|---|---|
| Opadry (ready-to-use film coating composition) | 12 |

### Example 6

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 150 + 1.5 |

Core comprising UDCA

| | |
|---|---|
| Ursodeoxycholic acid | 150 |
| L Arginine | 20 |
| Polyvinylpyrrolidone | 20 |
| Microcrystalline Cellulose | 80 |
| Magnesium Stearate | 4 |
| Talc | 4 |
| Hydroxypropylmethylcellulose | 12 |
| PEG 6000 | 4 |
| Titanium Dioxide | 2 |
| Eudragit L100 | 12 |
| Acetylated Monoglycerides | 2 |

STOP Layer

| | |
|---|---|
| Microcrystalline Cellulose | 30 |
| Corn starch | 10 |
| Crospovidone | 4 |
| Polyvinylpyrrolidone | 6 |

Second layer Comprising OCA

| | |
|---|---|
| Obeticholic acid | 1.5 |
| Microcrystalline Cellulose | 25 |
| Sodium Starch Glycollate | 4 |
| Colloidal Silicon Dioxide | 2 |
| Magnesium Stearate | 4 |

Gastric acid soluble layer

| | |
|---|---|
| Opadry (ready-to-use film coating composition) | 28 |

### Example 7

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 200 + 2.5 |

Core comprising UDCA

| | |
|---|---|
| Ursodeoxycholic acid | 200 |
| L Arginine | 30 |
| Polyvinylpyrrolidone | 25 |
| Microcrystalline Cellulose | 150 |
| Magnesium Stearate | 10 |
| Talc | 6 |
| Hydroxypropylmethylcellulose | 15 |
| PEG 6000 | 10 |
| Titanium Dioxide | 2 |
| Eudragit L100 | 24 |
| Acetylated Monoglycerides | 4 |

STOP Layer

| | |
|---|---|
| Microcrystalline Cellulose | 36 |
| Corn starch | 14 |
| Crospovidone | 6 |
| Polyvinylpyrrolidone | 6 |

Second layer Comprising OCA

| | |
|---|---|
| Obeticholic acid | 2.5 |
| Microcrystalline Cellulose | 30 |
| Sodium Starch Glycollate | 6 |
| Colloidal Silicon Dioxide | 4 |
| Magnesium Stearate | 6 |

Gastric acid soluble layer

| | |
|---|---|
| Opadry (ready-to-use film coating | 36 |

### Example 8

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 250 + 5 |

Core comprising UDCA

| | |
|---|---|
| Ursodeoxycholic acid | 250 |
| L Arginine | 40 |
| Polyvinylpyrrolidone | 30 |
| Microcrystalline Cellulose | 300 |
| Magnesium Stearate | 20 |
| Talc | 12 |
| Hydroxypropylmethylcellulose | 28 |
| PEG 6000 | 20 |
| Titanium Dioxide | 5 |
| Eudragit L100 | 44 |
| Acetylated Monoglycerides | 6 |

STOP Layer

| | |
|---|---|
| Microcrystalline Cellulose | 40 |
| Corn starch | 20 |
| Crospovidone | 8 |
| Polyvinylpyrrolidone | 8 |

Second layer Comprising OCA

| | |
|---|---|
| Obeticholic acid | 5 |
| Microcrystalline Cellulose | 40 |
| Sodium Starch Glycollate | 8 |
| Colloidal Silicon Dioxide | 4 |
| Magnesium Stearate | 8 |

Gastric acid soluble layer

| | |
|---|---|
| Opadry (ready-to-use film coating | 44 |

### Example 9

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 500 + 5 |

Core comprising UDCA

| | |
|---|---|
| Ursodeoxycholic acid | 500 |
| L Arginine | 45 |
| Polyvinylpyrrolidone | 34 |
| Microcrystalline Cellulose | 350 |
| Magnesium Stearate | 20 |
| Talc | 12 |
| Hydroxypropylmethylcellulose | 28 |
| PEG 6000 | 20 |
| Titanium Dioxide | 5 |
| Eudragit L100 | 44 |
| Acetylated Monoglycerides | 6 |

STOP Layer

| | |
|---|---|
| Microcrystalline Cellulose | 45 |
| Corn starch | 25 |
| Crospovidone | 8 |
| Polyvinylpyrrolidone | 8 |

Second layer Comprising OCA

| | |
|---|---|
| Obeticholic acid | 5 |
| Microcrystalline Cellulose | 60 |
| Sodium Starch Glycollate | 8 |
| Colloidal Silicon Dioxide | 8 |
| Magnesium Stearate | 8 |

Gastric acid soluble layer

| | |
|---|---|
| Opadry (ready-to-use film coating | 48 |

### Example 10

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 750 + 10 |

Core comprising UDCA

| | |
|---|---|
| Ursodeoxycholic acid | 750 |
| L Arginine | 50 |
| Polyvinylpyrrolidone | 40 |
| Microcrystalline Cellulose | 400 |
| Magnesium Stearate | 25 |
| Talc | 17 |
| Hydroxypropylmethylcellulose | 35 |
| PEG 6000 | 26 |
| Titanium Dioxide | 7 |
| Eudragit L100 | 50 |
| Acetylated Monoglycerides | 10 |

STOP Layer

| | |
|---|---|
| Microcrystalline Cellulose | 55 |
| Corn starch | 30 |
| Crospovidone | 10 |
| Polyvinylpyrrolidone | 10 |

Second layer Comprising OCA

| | |
|---|---|
| Obeticholic acid | 10 |
| Microcrystalline Cellulose | 80 |
| Sodium Starch Glycollate | 10 |
| Colloidal Silicon Dioxide | 10 |
| Magnesium Stearate | 10 |

Gastric acid soluble layer

| | |
|---|---|
| Opadry (ready-to-use film coating | 60 |

### Example 11

Ursodeoxycholic acid, L-arginine, 50% microcrystalline cellulose, 50% hydroxypropylmethylcellulose and polyvinylpyrrolidone are dry mixed and wet granulated in an appropriate granulator with sufficient purified water.

The wet granules are dried, milled, and blended with the remaining 50% microcrystalline cellulose, remaining 50% hydroxypropylmethylcellulose, talc, magnesium stearate, PEG 6000, titanium dioxide, Eudragit L100 and acetylated monoglycerides.

The final granule blend is compressed into tablet cores.

Microcrystalline cellulose, corn starch, crospovidone, and polyvinylpyrrolidone are added slowly to purified water and mixing is continued until the components are fully dispersed.

The dispersion is sprayed on to the tablet cores in a conventional coating pan until proper amount of STOP layer is deposited on the tablet cores.

Obeticholic acid is dissolved in purified water. After thorough mixing, microcrystalline cellulose, sodium starch glycollate, colloidal silicon dioxide, and magnesium stearate are added slowly, and mixing is continued until the components are fully dispersed. The suspension is sprayed on to the tablet cores covered with STOP layer in a conventional coating pan until the proper amount of Second layer comprising OCA is deposited.

Opadry is added slowly to purified water and mixing is continued until Opadry is fully dispersed. The solution is sprayed on to the tablet cores covered with STOP layer and second layer comprising OCA in a conventional coating pan until proper amount of Opadry is deposited on the tablets. A sample of the tablets is tested for gastric resistance, and the coating process is discontinued if the tablets meet the required criteria.

| | Quantity per |
|---|---|
| Ingredient | Tablet (mg) |
| API content, mg (UDCA + OCA) | 1000 + 10 |

Core comprising UDCA

| | |
|---|---|
| Ursodeoxycholic acid | 1000 |
| L Arginine | 70 |
| Polyvinylpyrrolidone | 50 |
| Microcrystalline Cellulose | 500 |
| Magnesium Stearate | 35 |
| Talc | 24 |
| Hydroxypropylmethylcellulose | 45 |
| PEG 6000 | 40 |
| Titanium Dioxide | 10 |
| Eudragit L100 | 70 |
| Acetylated Monoglycerides | 15 |

STOP Layer

| | |
|---|---|
| Microcrystalline Cellulose | 70 |
| Corn starch | 40 |
| Crospovidone | 12 |
| Polyvinylpyrrolidone | 12 |

Second layer Comprising OCA

| | |
|---|---|
| Obeticholic acid | 10 |
| Microcrystalline Cellulose | 100 |
| Sodium Starch Glycollate | 20 |
| Colloidal Silicon Dioxide | 15 |
| Magnesium Stearate | 15 |

Gastric acid soluble layer

| | |
|---|---|
| Opadry (ready-to-use film coating | 80 |

In order for a patient to receive the required daily dose of UDCA and OCA, the patient may take multiple tablets per day as illustrated in Examples 5-11 (for instance, three or four tablets of the formulation of Example 6), or a single tablet as illustrated in Examples 10-11.

The dissolution characteristics of the modified release solid dosage forms were evaluated for the tablet batches prepared according to Examples 5-11. Table 17 presents the median dissolution profile obtained for the batch manufactured according to Example 11, for both OCA and UDCA in the specified media.

TABLE 15
Values of the release rates of OCA and UDCA upon
dissolution of the claimed modified release solid dosage form, in %

| | API released (%) | | | | | | |
|---|---|---|---|---|---|---|---|
| | Time (min) | | | | | | |
| Parameter | 15 | 30 | 45 | 60 | 120 | 240 | 300 |
| OCA | 52 | 65 | 75 | 99 | 100 | 100 | 100 |
| UDCA | 0 | 0 | 0 | 0 | 23 | 48 | 99 |

### Example 12

A double-blind clinical study was conducted to evaluate the efficacy of the modified release solid dosage forms comprising UDCA and OCA in comparison with solid dosage forms comprising UDCA alone. The multilayer tablets corresponding to Embodiments 15 and 16 were used as modified release solid dosage forms comprising UDCA and OCA. The study assessed three treatment regimens in patients diagnosed with PBC:
1) A solid immediate release dosage form comprising UDCA at a dose of 15 mg/kg/day.
2) A modified release solid dosage form comprising UDCA and OCA at a dose of UDCA 15 mg/kg+OCA 5 mg per day.
3) A modified release solid dosage form comprising UDCA and OCA a dose of UDCA 15 mg/kg+OCA 10 mg per day.

The studies were conducted for 12 months, during which the quality of the treatment was assessed by measuring the biochemical and molecular biological markers (see above).

The efficacy of the treatment was evaluated on the basis of the Paris2 system, according to which the criteria for a satisfactory treatment are:
ALT level ≤3-fold upper limit of normal (ULN);
AST level ≤2-fold ULN;
normal bilirubin level.

The results are summarized in Table 16.

TABLE 18
Effectiveness of the PBC treatment using various regimens

| | | Number of | Number of |
|---|---|---|---|
| | | patients with | patients with |
| | | positive | positive |
| | Total | response to | response to |
| | number of | treatment | treatment |
| | patients | (number) | (%) |
| UDCA 15 mg/kg | 68 | 40 | 58.82 |
| UDCA 15 mg/kg + | 65 | 56 | 86.15 |
| OCA 5 mg | | | |
| UDCA 15 mg/kg + | 63 | 58 | 92.06 |
| OCA 10 mg | | | |

According to the data above, the treatment regimen using modified release solid dosage forms at the dose of UDCA 15 mg/kg+OCA 10 mg per day, was found to be the most effective (92.06% of the patients had a positive result of the treatment). The second most efficient was the treatment regimen using modified release solid dosage forms at the dose of UDCA 15 mg/kg+OCA 5 mg per day (86.15% of the patients had a positive result of the treatment). The regimen with the use of the drug comprising one API, UDCA, had the lowest effectiveness-58.82%.

### Example 13

A multicenter, randomized, double-blind, placebo-controlled, parallel-group, multidose study was conducted to determine the efficacy of the modified release solid dosage form comprising OCA and UDCA in the treatment of PBC.

The study included 165 men and women aged 18-75 years diagnosed with PBC who had been receiving a stable dose of UDCA for at least 6 months prior to screening. Patient weight ranged from 70 to 90 kg. The diagnosis of PBC was based on the presence of at least 2 of 3 key criteria:
(1) AMA titer greater than 1:40;
(2) abnormal alkaline phosphatase (Alk-p) levels [>1.5×upper limit of normal]; and
(3) liver histology compatible with PBC.

The patients were randomized to placebo group, receiving solid dosage forms comprising UDCA in the amount of 15 mg UDCA per 1 kg of the patient body weight, 1000 mg UDCA+10 mg OCA group (2 multilayer tablets according to Embodiment 18 (500 mg UDCA+5 mg OCA) per day for 12 weeks), and 1000 mg UDCA+20 mg OCA group (2 multilayer tablets according to Embodiment 16 (500 mg UDCA+10 mg OCA) per day for 12 weeks).

The 500 mg UDCA+5 mg OCA multilayer tablet according to Embodiment 18 comprises the outer layer comprising the gastric acid soluble layer-film coating, the second layer comprising 5 mg OCA, the third layer comprising the STOP layer, and the core comprising 500 mg UDCA. Similarly, the 500 mg UDCA+10 mg OCA multilayer tablet according to Embodiment 16 comprises outer layer comprising the gastric acid soluble layer-film coating, the second layer comprising 10 mg OCA, the third layer comprising the STOP layer, and the core comprising 500 mg UDCA.

The patients were assessed for changes in serum alkaline phosphatase (ALP) levels from baseline to Day 85 or early termination (ET).

TABLE 17
ALP (U/L) at baseline and on day 85

| | | | |
|---|---|---|---|
| | Placebo | UDCA + 10 mg | UDCA + 20 mg |
| Parameter | group | OCA group | OCA group |

Baseline

| | | | |
|---|---|---|---|
| Mean (SD) | 275.2 (102.7) | 294.4 (149.4) | 290.0 (123.6) |
| Median | 249.5 | 234.8 | 255.8 |

Day 85

| | | | |
|---|---|---|---|
| Mean (SD) | 270.7 (118.7) | 219.0 (113.5) | 225.0 (169.1) |
| Median | 234.5 | 177.5 | 187.5 |
| Mean (SD) | −2.6 (12.4) | −23.3 (17,1) | −24.0 (18.8) |
| percent change | | | |
| Median percent | −3.2 | −21.0 | −27.8 |
| change | | | |
| P-value | — | <0.0001 | <0.0001 |

The Table 17 data shows that compared with the placebo group, the UDCA+10 mg OCA and UDCA+20 mg OCA groups had a greater mean percentage decrease in ALP (−23.7%±17.8%, −24.7%+17.9%, respectively, compared with 2.6%±12.5%, p<0.0001). Thus, the study confirms the positive effect of modified release solid dosage forms comprising OCA and UDCA in the treatment of PBC in terms of changes in serum ALP.

### Example 14

A randomized clinical trial was conducted to determine the clinical results of the therapy using modified release solid dosage forms comprising UDCA and OCA in comparison with solid dosage forms comprising UDCA alone, in patients with PBC.

The study included 170 men and women aged 18-75 years diagnosed with PBC. The average patient weight was 78.9 kg. The diagnosis of PBC was based on the presence of at least 2 of 3 key criteria:
(1) AMA titer greater than 1:40;
(2) abnormal alkaline phosphatase (Alk-p) levels [≥1.5×upper limit of normal (ULN)]; and
(3) liver histology compatible with PBC.

The patients were randomized to placebo group, receiving solid dosage forms comprising UDCA in the amount of 15 mg UDCA per 1 kg of the patient body weight, 1000 mg UDCA+10 mg OCA group (2 multilayer tablets according to Embodiment 42 (500 mg UDCA+5 mg OCA) per day for 12 weeks), and 1000 mg UDCA+20 mg OCA group (2 multilayer tablets according to Embodiment 40 (500 mg UDCA+10 mg OCA) per day for 12 weeks).

The multilayer tablet according to Embodiment 42 comprising 500 mg UDCA+5 mg OCA comprises the outer layer comprising gastric acid soluble layer-film coating, the second layer comprising 5 mg OCA, the third layer comprising the enteric coating, and the core comprising 500 mg UDCA. Similarly, the multilayer tablet according to Embodiment 40 comprising 500 mg UDCA+10 mg OCA comprises the outer layer comprising the gastric acid soluble layer-film coating, the second layer comprising 10 mg OCA, the third layer comprising the enteric coating, and the core comprising 500 mg UDCA.

The primary endpoints were:
1) serum alkaline phosphatase less than 1.67 times the upper limit of normal (ULN) with a 15% reduction from baseline; and
2) total serum bilirubin within normal limits at study endpoints (85 days and 12 months).

Secondary outcomes included liver biochemistry parameters, including serum alanine aminotransferase (ALT), aspartate aminotransferase (AST), and gamma-glutamyl transpeptidase (GGT), as well as conjugated bilirubin.

Both endpoints were achieved in 52% of the patients in the 1000 mg UDCA+10 mg OCA and 1000 mg UDCA+20 mg OCA groups and in 22% of the patients in the placebo group. The results show that therapy with modified release solid dosage forms comprising UDCA and OCA was significantly superior to the therapy with UDCA alone in reducing serum ALT (median (MD)−15.63 IU/L; 95% confidence interval (CI), −21.59 to −9.68), AST (MD−6.63 IU/L; 95% CI, −11.03 to −2.24), and GGT (MD−131.30 IU/L; 95% CI, −177.52 to −85.08). However, there was no significant difference between the 1000 mg UDCA+10 mg OCA and 1000 mg UDCA+20 mg OCA groups and placebo group in reducing conjugated bilirubin (MD−0.06 mg/dL; 95% CI, −0.28 to 0.15; p=0.56). Overall, the results showed that the therapy with modified release solid dosage forms comprising UDCA and OCA was not significantly different from the therapy with UDCA alone in reducing bilirubin, but was statistically significantly superior to the therapy with UDCA alone in reducing liver biochemical parameters.

### Example 15

A randomized, double-blind, placebo-controlled study was conducted to determine the efficacy of modified release solid dosage forms comprising UDCA and OCA in patients with PBC.

The study included 77 men and women aged 18-75 years diagnosed with PBC. The average weight of patients was 80.1 kg. The diagnosis of PBC was based on the presence of at least 2 of 3 key criteria:
(1) AMA titer greater than 1:40;
(2) abnormal alkaline phosphatase (Alk-p) levels [>2×upper limit of normal (ULN)]; and
(3) liver histology compatible with PBC.

In addition, total bilirubin was <2.5×ULN.

The patients were randomized to placebo group, 500 mg UDCA+2.5 mg OCA group (Group 1, 1 multilayer tablet according to Embodiment 14 (500 mg UDCA+2.5 mg OCA) per day for the first 12 weeks) group, and 750 mg UDCA+5 mg OCA group (Group 2, 1 oral multilayer tablet (750 mg UDCA+5 mg OCA) per day for the first 12 weeks) group. After 12 weeks, provided there were no safety or tolerability limitations, the dose was titrated to 1000 mg UDCA+5 mg OCA (group 1, 2 multilayer tablets according to Embodiment 14 (500 mg UDCA+2.5 mg OCA) per day) and 1500 mg UDCA+10 mg OCA (group 2, 2 multilayer tablets (750 mg UDCA+5 mg OCA) per day), while the placebo group continued to administer placebo for an additional 12 weeks.

The 500 mg UDCA+2.5 mg OCA multilayer tablet comprises the outer layer comprising gastric acid soluble layer-film coating, the second layer comprising 2.5 mg OCA, the third layer comprising the STOP layer, and the core comprising 500 mg UDCA. Similarly, the 750 mg UDCA+5 mg OCA multilayer tablet comprises the outer layer comprising gastric acid soluble layer-film coating, the second layer comprising 5 mg OCA, the third layer comprising the STOP layer, and the core comprising 750 mg UDCA.

The primary efficacy endpoint was the change from baseline in serum ALP at week 24. Secondary endpoints included: changes in alanine aminotransferase, aspartate aminotransferase, gamma-glutamyl transferase, direct bilirubin, and total bilirubin levels.

Modified release solid dosage forms 500 mg UDCA+2.5 mg OCA administered to Group 1 did not show a significant biochemical response during the first 12 weeks of the study. At week 12, the decrease in ALP in Group 1 was not significantly different from the placebo. As at week 24, the ALP reduction in Group 1 was not significantly different from that in the placebo (mean difference=−78.29 [41.81] U/L, 95% CI−162.08 to 5.50; p=0.067).

In contrast, the Group 2 regimen resulted in a significant reduction in ALP, a biochemical marker of cholestasis, in the patients with PBC at week 24. Improvements in ALP were evident as early as 2 weeks after the start of the treatment and were maintained throughout the treatment. At week 12, the ALP reduction in Group 2 was significantly different from that in the placebo (mean difference −82.35 [33.39] U/L) (95% CI −149.11 to −15.59; p=0.017). At week 24, the reduction in ALP in group 2 was also significantly different from the placebo (mean difference-83.42 (40.34) U/L (95% CI-164.28 to −2.57; p=0.043). Titration in group 2 from 750 mg UDCA+5 mg OCA to 1500 mg UDCA+10 mg OCA did not lead to a further reduction in ALP.

Mean total and direct bilirubin values were within normal limits in all treatment groups during the first 12 weeks and generally fluctuated around their respective baseline values throughout the study. Mean AST and ALT values decreased during the first 12 weeks, with no statistically significant differences between the treatment groups. Median GGT values also decreased during the first 12 weeks, with greater reductions in Group 2 compared with both the placebo and Group 1. At the start of the second 12-week period, AST and ALT values remained below baseline, and AST reductions remained generally stable thereafter, whereas ALT values showed a slight further decrease. GGT values at the start of the second 12-week period were lower than baseline and remained generally stable during this period.

During the first 12 weeks, majority of the patients in each treatment group (88% to 96%) reported at least one treatment-associated adverse event. Most treatment-associated adverse events were mild to moderate in severity, but a higher proportion of patients in the treatment groups experienced severe treatment-associated adverse events than in the placebo group (group 1, 28%; group 2, 52%; placebo, 17%).

In summary, the study showed that the treatment of PBC with the modified release solid dosage forms comprising UDCA and OCA results in a sustained reduction in serum ALP levels. The treatment regimen was generally safe and well tolerated, with no evidence of worsening total bilirubin or other liver biochemistry parameters.

The studies relevant to the present disclosure may be supplemented as additional data become available.

The technical contribution of the present disclosure resides in the following:
use of the claimed modified release solid dosage form allows increasing the effectiveness of the treatment of PBC from 60% to more than 80%;
the claimed modified release solid dosage form is effective and suitable for use at all stages of PBC, including the early stages of the disease;
the claimed modified release solid dosage form is more convenient to use and is characterized by increased compliance, which is important in the treatment of such a severe chronic and incurable disease as PBC;
the claimed modified release solid dosage form incorporates a purpose-built structural configuration engineered to provide a sequential and time-partitioned release of 2 APIs. This prevents competition of the two acids for the transporters and ensures maximum bioavailability and effectiveness of both APIs.

The embodiments described herein are illustrative only and do not limit the scope of the disclosure.

1. A modified release solid dosage form for the treatment of primary biliary cholangitis, comprising:
(a) a core comprising ursodeoxycholic acid; and
(b) at least one layer comprising obeticholic acid,
wherein a weight ratio of ursodeoxycholic acid to obeticholic acid ranges from 2:1 to 1000:1.

2. The modified release solid dosage form according to claim 1, wherein the weight ratio of ursodeoxycholic acid to obeticholic acid ranges from 500:1 to 1000:1.

3. The modified release solid dosage form according to claim 2, wherein the weight ratio of ursodeoxycholic acid to obeticholic acid ranges from 700:1 to 1000:1.

4. The modified release solid dosage form according to claim 1, wherein the dosage form comprises the core and three layers, wherein the core comprises ursodeoxycholic acid and at least one of the layers comprises obeticholic acid.

5. The modified release solid dosage form according to claim 4, wherein the dosage form comprises an outer layer, a second layer comprising obeticholic acid, a third layer, and the core comprising ursodeoxycholic acid.

6. The modified release solid dosage form according to claim 5, wherein the outer layer comprises an enteric coating or a layer resistant to gastric acid.

7. The modified release solid dosage form according to claim 5, wherein the outer layer comprises a non-enteric coating or a gastric acid soluble layer.

8. The modified release solid dosage form according to claim 5, wherein the second layer comprises obeticholic acid in an amount of 1-50 mg.

9. The modified release solid dosage form according to claim 5, wherein the third layer comprises a STOP layer with a delayed dissolution or an enteric coating.

10. The modified release solid dosage form according to claim 9, wherein the dissolution time of the STOP layer is 1-2 hours.

11. The modified release solid dosage form according to claim 1, wherein the core comprises ursodeoxycholic acid in an amount of 100-1000 mg.

12. The modified release solid dosage form according to claim 1, wherein the core provides the modified release of ursodeoxycholic acid over a period of 1 to 6 hours.

13. The modified release solid dosage form according to claim 5, wherein the dosage form comprises:
(a) the outer layer comprising the enteric coating;
(b) the second layer comprising obeticholic acid;
(c) the third layer comprising the STOP layer; and
(d) the core comprising ursodeoxycholic acid.

14. The modified release solid dosage form according to claim 5, wherein the dosage form comprises:
(a) the outer layer comprising the gastric acid soluble layer;
(b) the second layer comprising obeticholic acid;
(c) the third layer comprising the enteric coating; and
(d) the core comprising ursodeoxycholic acid.

15. The modified release solid dosage form according to claim 13, wherein the dosage form is for the treatment of primary biliary cholangitis characterised by the following concentration level values of markers IL-6, Nf-kB, MCP-1/CCL2, and Bcl-2 in a patient sample:

| | |
|---|---|
| IL-6 | at least 3.0 pg/ml; |
| Nf-kB | at least 14.0 pg/ml; |
| MCP-1/CCL2 | at least 215 pg/ml; |
| Bcl-2 | at least 0.24 U/ml. |

16. The modified release solid dosage form according to claim 1, wherein primary biliary cholangitis is characterised by the following concentration level values of markers IL-6, Nf-kB, MCP-1/CCL2, Bcl-2, TNF-α, MDA in a patient sample:

| | |
|---|---|
| IL-6 | at least 3.0 pg/ml; |
| Nf-kB | at least 14.0 pg/ml; |
| MCP-1/CCL2 | at least 215 pg/ml; |
| Bcl-2 | at least 0.24 U/ml; |
| TNF-α | at least 0.14 hg/ml; |
| MDA | at least 4.3 nmol/ml. |
